(19) United States
(12) Patent Application Publication
(10) Pub. No.: US 2026/0327254 A1
(43) Pub. Date: Oct. 22, 2026

(54) SOLUTION-PROCESSABLE CHIRAL NANOSTRUCTURES, METHODS OF MAKING, AND USES THEREOF

(71) Applicant: University of Florida Research Foundation, Inc., Gainesville, FL (US)

(72) Inventors: Yunwei Charles CAO, Gainesville, FL (US); Tianyuan XIAO, Gainesville, FL (US)

(21) Appl. No.: 19/483,675

(22) PCT Filed: May 21, 2024
(86) PCT No.: PCT/US2024/030305

§ 371 Date: Nov. 12, 2025

Related U.S. Application Data
(60) Provisional application No. 63/468,406, filed on May 23, 2023.
(60) Provisional application No. 63/515,387, filed on Jul. 25, 2023.

(51) Int. Cl. C07J 9/00; G01N 23/04; H10F 77/14; H10H 20/812

(52) U.S. Cl. CPC C07J 9/005; G01N 23/04; H10F 77/143; H10H 20/812

(57) ABSTRACT

In one aspect, the disclosure relates to methods for preparing compositions comprising a chiral nanostructure, wherein the nanostructure comprises a semiconductor material, the method comprising admixing a chiral ligand, optionally a metal salt, and at least one inorganic-material forming precursor in a nonpolar organic solvent. Also disclosed herein are compositions and articles comprising the chiral nanostructures and methods for performing biological imaging using the chiral nanostructures. This abstract is intended as a scanning tool for purposes of searching in the particular art and is not intended to be limiting of the present disclosure.

### CROSS REFERENCE TO RELATED APPLICATIONS

This application claims the benefit of U.S. Provisional Application No. 63/468,406 filed on May 23, 2023, and of U.S. Provisional Application No. 63/515,387 filed on Jul. 25, 2023, each of which is incorporated herein by reference in its entirety.

### STATEMENT REGARDING FEDERALLY SPONSORED RESEARCH OR DEVELOPMENT

This invention was made with government support under Grant Number 1710509, awarded by the National Science Foundation. The government has certain rights in the invention.

### BACKGROUND

Chirality is one of the most fundamental properties within various objects in the universe. Examples range from the parity violation in the weak interaction to biological systems on earth. At 1848, the theory of molecular chirality and optical activity was proposed by Louis Pasteur after experiments on the sodium ammonium salt of the tartaric acid crystal, during which he observed that two opposite crystal shapes were formed during the crystallization process. Ever since that discovery, chiral molecules have attracted large attention in organic chemistry and biochemistry. However, chirality is not solely limited to organic molecules. Recently, with the development of nano science, researchers have found asymmetric particles having sizes within the orders of magnitude of electromagnetic wavelengths. Thus, chiral inorganic nanostructures, or nanostructures having chiroptical activity were observed.

Compared with conventional chiral molecules like organic small molecules and large biomacromolecules, chiral inorganic nanostructures can exhibit much stronger chiroptical activity, therefore exhibiting great potential for use in various applications including bioimaging, electrooptical devices, and sensing. These nanostructures can further be chemically tailored to exhibit exceptional optoelectronic properties for making structurally flexible and highly efficient optoelectronic and photonic devices. This manufactural advance allows for a systematic study of the impact of chirality on light-matter interactions in soft materials and the further exploitations for the simultaneous and quantum mechanical control of charge, spin, and light.

Similar to chiral organic molecules, the chirality of inorganic nanostructures originates from the asymmetricity in either shape or atomic arrangement. The pioneering work in this field was published by Whetten and Schaaff; in their research, small gold clusters capped with L-glutathione molecules showed circular dichroism (CD) activity in electronic transitions. Three possible mechanisms were suggested: (1) intrinsic chiral atomic arrangement guided by the strong bonding of chiral ligand; (2) achiral cores interacting with chiral arrangement pattern of surface ligands; (3) direct electronic interaction between the core electrons and the chiral centers of the ligand molecules. Later researchers discovered that the formation of chiral shaped nanoparticles or achiral nanoparticles assemblies in chiral arrangement with the asymmetric size within the order of excitation wavelength are also possible methods to generate chiroptical activity.

Based on these discoveries, many experimental and theoretical works have been reported on chiral inorganic nanoparticles, especially on Au nanoparticles. With chiral ligands, the strong thiol-gold bond induces chiral atomic arrangements on the surface of gold clusters and influences the electronic states of the nanoparticle. Both effects result in chiroptical activity. For example, Dolamic and co-workers synthesized Au₃₈ clusters with intrinsic chirality by reducing tetrachlorauric acid in the presence of L-/D-glutathionate. This work proved that a chiral surface atom arrangement was feasible even for materials with highly symmetric crystal structures. Another example of nanoparticles with intrinsic chirality was carried out by Wu et al., by applying a vapor transport method to synthesize chiral hexagonal nanowebs of chromium silicide. The hexagonal nanowebs showed chirality on both atomic arrangement and entire macroscopic shape. However, the sample itself was probably a racemic mixture and, thus, no chiroptical activity measurement was reported. Very recently, the concept of enantioselective synthesis for intrinsically chiral inorganic nanoparticles was reported by Ben-Moshe et al., of which the major idea is applying high concentration of enantiomerically pure chiral molecules that can bind strongly to the surface of nanoparticles into nucleation solutions during nanoparticles synthesis. In their work, chiral HgS nanoparticles were synthesized by precipitate Hg²⁺ and S²⁻ in aqueous phase in the presence of L- or D-penicillamine. The thiol groups on penicillamine bind strongly to Hg, inducing chiral perturbation.

Another mechanism of generating chiroptical activity on nanoparticles is by utilizing the interaction between achiral nanoparticles and chiral molecules. Recently, researchers showed that the chiroptical activity could be induced by attaching chiral molecules to the surface of achiral noble metal nanoparticle. For instance, chiroptical activity was reported to be induced by the interaction between gold nanoparticles and chiral molecules without direct bonding. This effect suggested a new concept of chiral molecule sensing, which may have potential applications for chiral bio-molecule sensors. Also, because of the tetrahedral geometry within CdTe crystal structure, the chirality in tetrahedral molecules with four different substituents could be investigated.

Besides core-ligand interactions, examining chiral shapes with the asymmetric size at the order of chiroptically active wavelength is another approach for preparing chiral nanoparticles. Two types of methods are commonly applied: top down and bottom up. The top down approach is straightforward, as the nanoparticles with designed chiral shapes are prepared using techniques including focused ion or electron beam lithography, glancing angle deposition, multi-photon direct laser writing, and holographic lithography. For the bottom up synthesis, wet chemistry methods are most widely applied. The major challenge of wet chemical methods is the selectivity of handedness. Recently, enantioselective synthesis of TiO₂ was carried out by Liu et al., in which enantiomerically pure amorphous TiO₂ nanofibers were prepared with titanium-organic precursor and C18-D/L-glutamic acid as ligand in aqueous phase. By calcining the nanofiber, all the organic compounds were removed and pure anatase TiO₂ was obtained. Both the amorphous and anatase TiO₂ nanofiber showed chiroptical activity. This work demonstrated that, attributed to the electronic transition, the chemically synthesized chiral shaped nanoparticle without chiral organic ligand could exhibit chiroptical activity. Cysteine has also been applied as an ligand in gold nanoparticle synthesis, which different crystal faces were exposed in a controlled fashion, which lead to an overall chiral shape, giving chiral optical activity. Beside gold nanoparticles, cysteine has also been used as surface ligand to synthesize Co₃O₄ magnetic nanoparticles, which achieved chiral optical activity in visible wavelength range, as well as optical activity Raman spectrum, which indicating the asymmetric vibration in the nanoparticles.

In addition to monodisperse nanoparticles, the chiral assemblies of achiral nanoparticles have also been shown to exhibit chiroptical activity. For example, the chiroptical effect may extend over the nanometer scale within plasmonic nanoparticle assemblies. Mastroianni and co-workers made a chiral pyramidal gold nanoparticle assembly with DNA as ligand, but no chiroptical characterization was performed. Later, Kuzyk and co-workers demonstrated that, using a DNA Origami scaffold, helical gold nanoparticle assemblies could be achieved in aqueous phase with chiroptical activity.

Chiroptical activity can be generated with combined mechanisms described above. For instance, Ben-Moshe and co-workers synthesized chiral Te nanoparticles by using chiral biomolecules as ligand. Both a chiral atomic arrangement and shape were observed. Moreover, the combination of chiral atomic arrangement and shape showed a higher chiroptical activity than intrinsic chirality itself. In another example, chiral structures were synthesized by assembling CdTe nanoclusters guided by circular polarized light. The chiroptical activity was reported to be induced by both inter-cluster interaction and the chiral shape of the assembly. With asymmetric shapes, nanostructures synthesized by lithography could achieve strong chiral emission.

Thus, different methods have been developed for preparing chiroptically-active nanoparticles, but most of the studies were reported to be conducted in aqueous systems. The major disadvantage for nanoparticles' synthesis in aqueous systems is the relatively poor shape and size distribution as compared to those in non-polar system. Particularly, II-VI semiconductor nanoparticles, such as CdS, CdSe, and CdTe, are widely known for their matured synthesis in non-polar organic systems. For example, two-dimensional II-VI semiconductor nano sheets and ribbon have been frequently reported. With at least one dimension in the quantum size range, these materials present strong one-dimensional quantum confinement properties and may find interesting applications in bio-imaging labeling, solar cells, light emitting diodes, and the like. Moreover, having at least one dimension in bulk size enables convenient size and shape manipulation. However, the chiral synthesis and characterization of such systems has not been conducted. Therefore, the development of facile synthesis methodologies for chiral two-dimensional II-VI semiconductor materials in non-polar organic systems with narrow shape and size distribution is of great importance.

Despite advances in chiral nanosystem and nanoparticle research, there is still a scarcity of methods that to synthesize chiral nanostructures that do not rely on aqueous solutions and that are suitable for the synthesis of II-VI semiconductor nanostructures. These needs and other needs are satisfied by the present disclosure.

### SUMMARY

In accordance with the purpose(s) of the present disclosure, as embodied and broadly described herein, the disclosure, in one aspect, relates to methods for preparing compositions comprising a chiral nanostructure, wherein the nanostructure comprises a semiconductor material, the method comprising admixing a chiral ligand, optionally a metal salt, and at least one inorganic-material forming precursor in a nonpolar organic solvent. Also disclosed herein are compositions and articles comprising the chiral nanostructures and methods for performing biological imaging using the chiral nanostructures.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIGS. 1A-1B show a scheme of linear and elliptical circular polarized light. (FIG. 1A) Linear polarized light, and (FIG. 1B) elliptical circular polarized light

FIGS. 2A-2B show UV-Vis spectra of aliquots of CdSe nanostructures synthesized with C₁₀Cho. (FIG. 2A) CdSe-390, and (FIG. 2B) CdSe-460.

FIGS. 3A-3B show UV-Vis and CD spectra of CdSe nanostructures synthesized with C₁₀Cho. (FIG. 3A) CdSe-390, and (FIG. 3B) CdSe-460.

FIG. 4 shows UV-Vis spectra of aliquots of CdS-324 nanostructures synthesized with C₁₀Cho.

FIG. 5 shows UV-Vis and CD spectra of CdS-324 nanostructures synthesized with C₁₀Cho.

FIGS. 6A-6B show UV-Vis spectra of aliquots of CdTe nanostructures synthesized with C₁₀Cho. (FIG. 6A) CdTe-425, and (FIG. 6B) CdTe-500.

FIGS. 7A-7B show UV-Vis and CD spectra of CdTe nanostructures synthesized with C₁₀Cho. (FIG. 7A) CdTe-425, and (FIG. 7B) CdTe-500.

FIG. 8 shows the molecular structures of the cholesteryl ligand and the lithocholyl ligand.

FIGS. 9A-9B show UV-Vis and CD spectra of CdS nanostructures synthesized with (FIG. 9A) lithocholyl ligand, and (FIG. 9B) C₁₀Cho ligand.

FIGS. 10A-10D show CD spectra of CdSe-390 synthesized with series of cholesteryl ligands. (FIG. 10A) C₆Cho and C₇Cho, (FIG. 10B) C₈Cho and C₉Cho, (FIG. 10C) C₁₀Cho, and C₁₁Cho, and (FIG. 10D) C₁₂Cho and C₁₃Cho.

FIGS. 11A-11D show CD spectra of CdS-324 synthesized with series of cholesteryl ligands. (FIG. 11A) C₆Cho and C₇Cho, (FIG. 11B) C₈Cho and C₉Cho, (FIG. 11C) C₁₀Cho, and C₁₁Cho, and (FIG. 11D) C₁₂Cho and C₁₃Cho.

FIGS. 12A-12C show CD spectra of CdTe-425 synthesized with series of cholesteryl ligands. (FIG. 12A) C₈Cho and C₉Cho, (FIG. 12B) C₁₀Cho, and C₁₁Cho, and (FIG. 12C) C₁₂Cho and C₁₃Cho.

FIGS. 13A-13B show (FIG. 13A) IR spectrum of pure C₉Cho and C₁₀Cho ligands (FIG. 13B) VCD spectrum of pure C₉Cho and C₁₀Cho ligands.

FIGS. 14A-14B show (FIG. 14A) IR spectrum of pure C₉Cho and C₁₀Cho ligands and (FIG. 14B) VCD spectrum of pure C₉Cho and C₁₀Cho ligands.

FIGS. 15A-15D show TEM images of purified chiral nanostructure (FIG. 15A) CdSe—C9, and (FIG. 15B) CdSe—C10. (FIG. 15C) Predicted 3D model of CdSe chiral nanostructure with different chirality. Left two images: left hand chirality, and right two images: right hand chirality. (FIG. 15D) TEM image of CdSe nanostructure synthesized with myristic acid.

FIGS. 16A-16B show TEM tilting imaging with 3D model for (FIG. 16A) CdSe—C9, and (FIG. 16B) CdSe—C10 at 0 degree and 30 degree.

FIGS. 17A-17C show (FIG. 17A) UV-Vis spectrum of CdSe—C9 synthesized with different guest molecules, (FIG. 17B) CD spectrum of CdSe—C9 synthesized with different guest molecules, and (FIG. 17C) molecular structure of the different guest molecules.

FIGS. 18A-18D show TEM images of CdSe—C9 synthesized with (FIG. 18A) Squalene, (FIG. 18B) Toluene, (FIG. 18C) Tetralin, and (FIG. 18D) ethyl cyclohexane.

FIG. 19 shows the molecular structures of epi-cholesterol, cholesterol, and ent-cholesterol.

FIGS. 20A-20B show (FIG. 20A) a molecular structure showing the a face and B face of cholesterol molecule and (FIG. 20B) reaction pathway for epi-cholesterol synthesis.

FIGS. 21A-21B show (FIG. 21A) CD spectrum of CdSe—C9, CdSe—C10, CdSe-epi-C9, and CdSe-epi-C10 and (FIG. 21B) a zoomed in view of CD spectrum for CdSe-epi-C9 and CdSe-epi-C10.

FIG. 22 shows a CD spectrum of CdSe—C9 and CdSe—C10 with different Mn ions doping level.

FIG. 23 shows a CD spectrum of CdSe—C9 and CdSe—C10 with different Pb ions doping level.

FIGS. 24A-24B show (FIG. 24A) a schematic illustration of strain release by doping ions with different radius than cadmium. (Top) Undoped crystal, (Bottom Left) doped with Mn²⁺ ions, which have smaller radius than Cd²⁺, (bottom middle) co-doped with Mn²⁺ and Pb²⁺ ions and (Bottom Right) doped with Pb²⁺ ions, which have larger radius than Cd²⁺; and (FIG. 24B) UV-Vis absorption spectra of Chiral CdSe—C9 and CdSe—C10 nanocrystal co-doped with Mn²⁺ and Pb²⁺ ions.

FIG. 25 is a schematic of the design of chiral ligands. A chiral ligand consists of three major moieties: an anchor group (A), a cross-linker (CL), and a chiral center (R). The anchor group can be any functional group that can form a covalent bond or a coordination bond with atoms on the surface of nanostructures, where ‘n’ denotes the number of methylene units in the alkyl chain. Depending on the types of anchor groups, these ligands can be classified as L-, X- and Z-type ligands, which contribute two, one, or zero electron(s) to a bonding orbital formed with atoms on the surface of nanostructures, respectively. The chiral center can be any non-polar chiral molecule framework that provides the chirality of the ligand. The cross-linker group can be any organic structure that connects the anchor group and chiral center together.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION

In one aspect, disclosed herein is a method for preparing a composition that includes a chiral nanostructure, where the nanostructure includes a semiconductor material. In a further aspect, the method includes the step of admixing a chiral ligand and at least one inorganic-material forming precursor in a nonpolar organic solvent.

In some aspects, the chiral ligand has a structure according to Formula I:
wherein A is selected from

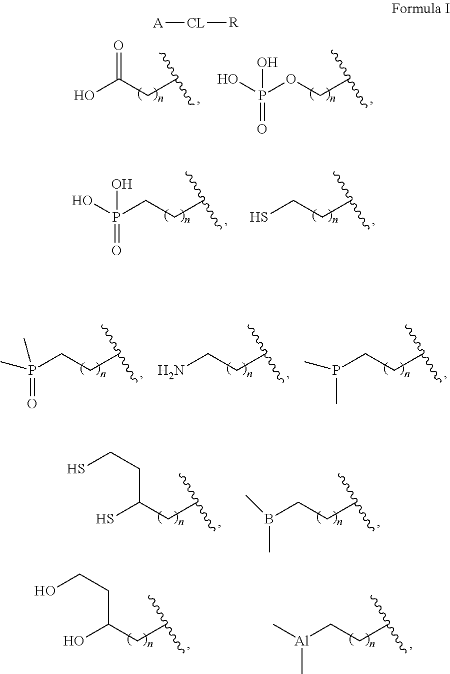

or any combination thereof
wherein n is from 1 to 14;
wherein CL is selected from

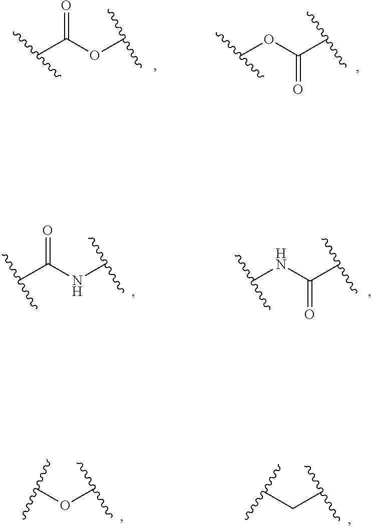

or any combination thereof; and
wherein R is selected from

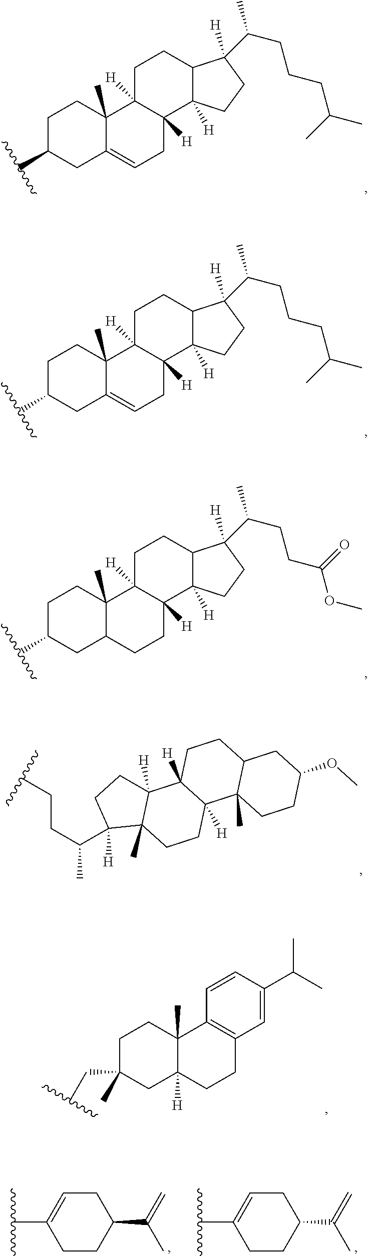

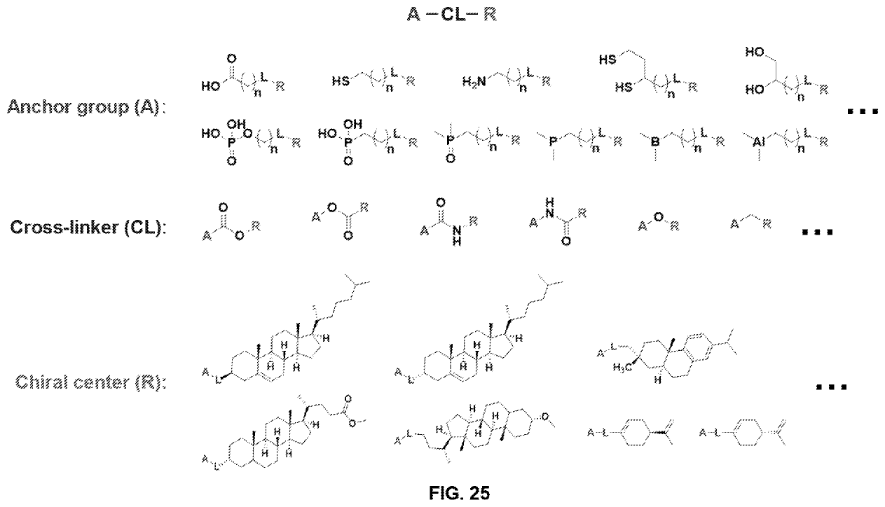

or any combination thereof. Exemplary compounds and substituents according to Formula I are shown in FIG. 25.

In a further aspect, the chiral ligand can be a carboxylic acid-functionalized cholesterol derivative of Formula II:

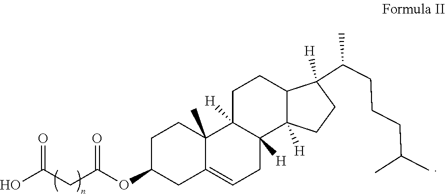

In some aspects, n is odd, and in other aspects, n is even.

In one aspect, the inorganic material can be a semiconductor material. In a further aspect, the method further includes the step of admixing a metal salt with the chiral ligand and the at least one inorganic-material forming precursor. In any of these aspects, the metal salt can include a Cd, Cu, Pb, Hg, or Zn salt, or any combination thereof. In some aspects, the metal salt includes a group 12 metal salt such as, for example a cadmium salt. In one aspect, the cadmium salt can be CdAC₂·2H₂O.

In another aspect, the inorganic-material forming precursor can be a chalcogen or a chalcogen precursor. In a further aspect, the chalcogen can be selected from sulfur, selenium, tellurium, or any combination thereof. In an alternative aspect, the chalcogen precursor can be trioctylphosphine-Te (TOP-Te), tributylphosphine-Se (TBP-Se), or any combination thereof. In one aspect, the nonpolar organic solvent can be 1-octadecene (ODE).

In another aspect, the method includes doping the composition with a second metal such as, for example, Pb, Mn, or any combination thereof.

Also disclosed herein are compositions including a chiral nanostructure prepared by a disclosed method. In another aspect, n in Formula I can be even and the nanostructure can be levorotatory. In an alternative aspect, n in Formula I can be odd, and the nanostructure can be dextrorotatory. In any of these aspects, the nanostructure can include one or more nanoparticles, a cluster of nanoparticles, a two-dimensional nanostructure, or any combination thereof.

In an aspect, the nanostructure can be or include CdS, CdSe, CdTe, or any combination thereof. In a further aspect, the nanostructure can have an absorption maximum at between about 324 nm and about 500 nm, or between about 330 nm and about 460 nm, or between about 380 nm and about 420 nm. In some aspects, the nanostructure can be a two-dimensional nanostructure having a thickness of from about 1.22 nm to about 1.83 nm, from about 1.3 to about 1.8 nm, or from about 1.3 to about 1.7 nm. In an alternative aspect, the nanostructure can have a helical shape.

Furthermore, disclosed herein are articles including the disclosed compositions and nanostructures such as, for example, solar cells, light emitting diodes, other electronic components, and the like.

Also disclosed herein is a method for detecting a target analyte in a biological sample, the method including at least the step of contacting the biological sample with a disclosed composition and imaging the sample. In a further aspect, the biological sample can be or include cells, tissues, an organ, or a fluid sample collected from a subject.

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “a cholesterol derivative,” “a methylene unit,” or “an ion,” include, but are not limited to, mixtures or combinations of two or more such cholesterol derivatives, methylene units, or ions, and the like.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the terms “optional” or “optionally” means that the subsequently described event or circumstance can or cannot occur, and that the description includes instances where said event or circumstance occurs and instances where it does not.

“Optical activity” is used to describe the interaction of chiral materials with polarized light. In a solution, the levorotatory-form, or (−)-form of an optical isomer rotates the plane of a beam of linearly polarized light counterclockwise. The dextrorotatory form, or (+)-form of an optical isomer does the opposite.

A “chiral nanostructure” as used herein is an object exhibiting chirality that consists of at least one nanostructured component, such as individual nanoparticles, nanofibers, nanoribbons, and their assemblies.

Unless otherwise specified, temperatures referred to herein are based on atmospheric pressure (i.e. one atmosphere).

Now having described the aspects of the present disclosure, in general, the following Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1: Materials and Methods

### Chemicals

Cadmium acetate dihydrate (98%), sulfur powder (99.999%), selenium powder (99.99%), tellurium (99.997%), succinic acid (99%), glutaric acid (99%), adipic acid (99%), pimelic acid (98%), suberic acid (98%), azelaic acid (98%), sebacic acid (99%), dodecanedioic acid (99%), oleic acid (OA, 90%), 1-octadecene (ODE, 90%), tributylphosphine (TBP, 97%), trioctylphosphine (TOP, 90%), triphenylphosphine (TPP, 95+%), diisopropyl azodicarboxylate (DIAD, 98%), and iron (III) chloride hexahydrate (97%) were purchased from Sigma Aldrich. Tridecanedioic Acid (96.0+%) and nonanedicarboxylic Acid (97+%) were purchased from TCI America. Cholesterol (95%) was purchased from Alfa Aesar. All other solvents were purchased from Fisher Scientific International, Inc. All chemicals were used without further purification.

### Synthesis of Chiral Ligands

Carboxylic acid functionalized cholesterol derivatives. Carboxylic acid functionalized cholesterol derivatives were synthesized as scheme 1. In a typical synthesis, 10.0 mmol cholesterol, 30 mmol dicarboxylic acid with specific hydrocarbon chain length, and 0.1 mmol FeCl₃·6H₂O were mixed in a 100 mL three neck-flask. The flask was then connected to the schlenk line, and heat was applied with a heating mantle to melt the solid. After heating for 8 hours with frequent pump to remove any water produced from the reaction, the flask was cooled down naturally to room temperature. The brownish solid was then dissolved with dichloromethane and dried with MgSO₄. The insoluble unreacted dodecanedioic acid was removed together with MgSO₄ by filtration, and the solvent was evaporated by rotovap. The residue was purified by a silica gel column chromatography eluting with Hexanes/Ethyl acetate (10:1, v/v) to give cholesteryl dicarboxylic acid in 70% yield as white solid. Depending on the length of dicarboxylic acid used for the chiral ligand synthesis, these chiral ligands were abbreviated as CₙCho, while n is the length of the alkyl chain of the dicarboxylic acid (Table 1). NMR results are shown in Table 2.

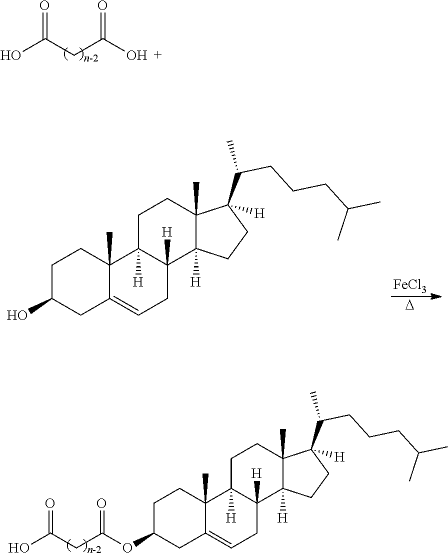

Scheme 1: Synthesis route for carboxylic acid functionalized cholesterol derivatives. n as shown is 2 but can be another even or odd number.

TABLE 1
Abbreviations for different chiral ligands

| Molecules | Abbreviations |
|---|---|
| 6-cholesteroxy-6-oxohexanoic acid | C₆Cho |
| 7-cholesteroxy-7-oxoheptanoic acid | C₇Cho |
| 8-cholesteroxy-8-oxooctanoic acid | C₈Cho |
| 9-cholesteroxy-9-oxononanoic acid | C₉Cho |
| 10-cholesteroxy-10-oxodecanoic acid | C₁₀Cho |
| 11-cholesteroxy-11-oxoundecanoic acid | C₁₁Cho |
| 12-cholesteroxy-12-oxododecanoic acid | C₁₂Cho |
| 13-cholesteroxy-13-oxotridecanoic acid | C₁₃Cho |

TABLE 2
¹H NMR results for synthesized ligands

| Molecules | ¹H NMR (300 MHz, CDCl₃) δ |
|---|---|
| C₆Cho | 5.38 (1H), 4.56-4.68 (1H), 2.22-2.35 (6H), 1.76-2.09 (5H), |
| | 0.87-1.69 (37H), 0.67 (3H) |
| C₇Cho | 5.37 (1H), 4.56-4.68 (1H), 2.25-2.38 (6H), 1.77-2.08 (5H), |
| | 0.85-1.70 (39H), 0.69 (3H) |
| C₈Cho | 5.36 (1H), 4.55-4.64 (1H), 2.23-2.38 (6H), 1.75-2.08 (5H), |
| | 0.86-1.72 (41H), 0.66 (3H) |
| C₉Cho | 5.39 (1H), 4.57-4.65 (1H), 2.23-2.35 (6H), 1.75-2.08 (5H), |
| | 0.86-1.71 (43H), 0.67 (3H) |
| C₁₀Cho | 5.34 (1H), 4.53-4.64 (1H), 2.25-2.38 (6H), 1.76-2.07 (5H), |
| | 0.85-1.71 (45H), 0.66 (3H) |
| C₁₁Cho | 5.36 (1H), 4.55-4.65 (1H), 2.24-2.38 (6H), 1.75-2.05 (5H), |
| | 0.86-1.70 (47H), 0.67 (3H) |
| C₁₂Cho | 5.35 (1H), 4.55-4.66 (1H), 2.24-2.37 (6H), 1.78-2.08 (5H), |
| | 0.85-1.68 (49H), 0.66 (3H) |
| C₁₃Cho | 5.37 (1H), 4.54-4.65 (1H), 2.22-2.38 (6H), 1.75-2.05 (5H), |
| | 0.86-1.70 (51H), 0.67 (3H) |

Methyl lithocholic acid. 5 mmol cholesterol and 15 mmol NaH were charged in a three-neck flask. The flask was then connected to a schlenk line and pumped for 3 times to achieve an oxygen and water free system, then 200 ml anhydrous THF was injected into the flask. After stirring for 10 minutes, 8 mmol Iodomethane was added drop wise, and the reaction was refluxed for 10 hours, before quenched with 1 M HCl. The mixture was then washed with water, the organic phase was collected, and aqueous phase was extracted with ethyl acetate for 3 times. The organic phase was combined and dried with MgSO₄, followed by filtration and solvent removal by rotovap. The product was purified by a silica gel column chromatography eluting with Hexanes/Ethyl acetate (20:1, v/v) to give methyl-lithocholic acid as white solid. 1H NMR (300 MHZ, CDCl₃): δ 3.35 (3H), 3.12-3.20 (1H), 2.19-2.44 (2H), 0.96-1.96 (26H), 0.91 (3H), 0.63 (3H).

Epi-cholesteryl ligand synthesis. The synthesis of epi-cholesteryl ligands was divided into two steps. The first step was to synthesize epi-cholesterol. Mitsunobu reaction was conducted to flip the hydroxyl group on cholesterol ring. Typically, 10 mmol of cholesterol, 10 mmol of 4-nitrobenzoic acid, and 20 mmol of triphenylphosphine was charged in a three neck-flask, then vacuumed and purged by Ar for three times to eliminate the oxygen and water in the system. Then, 50 mL of anhydrous THF was added to the flask. The mixture was stirred at room temperature for 3 min until a clear solution was formed. The flask was cooled to 0° C., and 20 mmol of diisopropyl azodicarboxylate was added dropwise to the reaction system. After reaction at room temperature for 4 h, the mixture was extracted by DCM and water, and organic phase was collected and dried with MgSO₄, followed by filtration and solvent removal by rotovap. The mixture was then purified by silica gel chromatography eluting with Hexane/Ethyl acetate (100:1, v/v) to give epi-cholesteryl nitrobenzoate as yellow solid. Then the solid was dissolved in a mixture of ethanol, chloroform, and water (2:1:1) to form a clear solution, and 70 mmol of NaOH was added to the mixture. The solution was stirred at room temperature overnight and extracted by ethyl acetate and water. The organic phase was collected and dried by MgSO₄ followed by filtration and solvent removal by rotovap. The product was purified by silica chromatography eluting with hexane/ethyl acetate (10:1, v/v) to give epi-cholesterol in 50% as white solid.

The second step of ester formation reaction with di-carboxylic acid and epi-cholesterol was identical to the reaction described in 4-2-2-1 except in a smaller reaction scale.

9-epicholesteroxy-9-oxononanoic acid (Epi-C9Cho): ¹H NMR (300 MHZ, CDCl₃): δ 5.20 (1H), 4.94 (1H), 2.18-2.50 (6H), 1.74-2.05 (7H), 0.85-1.68 (43H), 0.68 (3H). 10-epicholesteroxy-10-oxodecanoic acid (Epi-C₁₀Cho): ¹H NMR (300 MHZ, CDCl₃): δ 5.26 (1H), 5.00 (1H), 2.18-2.50 (7H), 1.74-2.04 (6H), 0.85-1.68 (43H), 0.68 (3H).

### Synthesis of Chiral CdS Nanostructures

CdS with first absorption peak at 324 nm. The asymmetric CdS nanostructures were synthesized using a one-pot method. Typically, 0.1 mmol CdAC₂·2H₂O, 0.2 mmol selected chiral ligand, 0.05 mmol sulfur, and 5.0 g ODE were mixed in a three neck-flask. The flask was connected to a schlenk line and pumped at room temperature for 3 times to remove oxygen and water in the system. Then, the reaction was heated to 120° C., and pumped for 50 min. During this process, the chiral ligand will react with CdAC₂·2H₂O, producing Cd(CₙCho)₂ as a precursor for the growth of nanostructure. After vacuum at 120° C. for 50 min, the reaction was heated to 180° C. and stayed for 50 min under argon flow, and aliquots were taken to monitor the reaction. The reaction was allowed to cool to room temperature naturally, and the resulting product was purified by adding polar solvent to precipitate, and re-dispersed in hexane for further analysis.

CdS with first absorption peak at 370 nm. The asymmetric CdS nanostructures was synthesized using one-pot method. Typically, 0.10 mmol CdAC₂·2H₂O, 0.20 mmol selected chiral ligand, 1.00 g 0.05 mmol/g S-ODE, and 4.00 g ODE were mixed in a three neck-flask. (S-ODE was prepared by mixing 8 mg sulfur with 5.00 g ODE, and then sonicate with a bench top sonicator for 20 min) The flask was connected to a schlenk line and pumped at room temperature for 3 times to remove oxygen and water. Then, the mixture was heated to 180° C. and stayed for 50 min under argon flow. Aliquots were taken at different time intervals to monitor the reaction. The resulting product was purified by adding acetone to precipitate and centrifuging at 3300 rpm for 2 min to separate, then re-dispersed in hexane for further usage.

### Synthesis of Chiral CdSe Nanostructures

CdSe with first absorption peak at 389 nm. The asymmetric CdSe nanostructures with first absorption at 389 nm was synthesized using an injection method. Typically, 0.10 mmol CdAC₂·2H₂O, 0.20 mmol selected chiral ligand, and 4.00 g ODE were charged in a three neck-flask. The flask was connected to a schlenk line and vacuumed at room temperature for 3 times to remove oxygen and water. Then, the reaction was heated to 120° C. and pumped for 10 min. After recharging the flask with Ar, the reaction solution was cooled to 100° C., and 100 μL of 1M TBP-Se was injected into the flask. The flask was maintained at 100° C. for 18 hours under argon flow, and aliquots were taken during the process. After the reaction was done, the solution was cooled naturally to room temperature, and the nanoparticles was separated by adding acetone to precipitate and centrifuging at 3300 rpm for 2 min. The obtained nanoparticles were re-dispersed in hexane for further usage.

CdSe with first absorption peak at 460 nm. The asymmetric CdSe nanostructures with first absorption at 460 nm was synthesized using a one pot method. Typically, 0.10 mmol CdAC₂·2H₂O, 0.20 mmol selected chiral ligand, 0.05 mmol Se powder, and 4.00 g ODE were charged in a three neck-flask. The flask was connected to a schlenk line and pumped at room temperature for 3 times to remove oxygen and water. Then the reaction was heated to 180° C. for 50 min under argon flow, and aliquots were taken to monitor the reaction. The resulting product was purified by adding acetone to precipitate and centrifuging at 3300 rpm for 2 min to separate, then re-dispersed in hexane for further usage.

### Synthesis of Chiral CdTe Nanostructures

CdTe with first absorption peak at 425 nm. The asymmetric CdTe nanostructures with first absorption at 460 nm was synthesized using an injection method. Typically, 0.10 mmol CdAC₂·2H₂O, 0.20 mmol selected chiral ligand, and 4.00 g ODE were charged in a three neck-flask. The flask was connected to a schlenk line and pumped at room temperature for 3 times to remove oxygen and water. Then the reaction was heated to 120° C. under argon flow and 100 μL of 1 M TOP-Te was injected into the flask (TOP-Te was synthesized by dissolving Te powder in TBP in a glove box). The reaction was maintained at 120° C. for 45 min under argon flow, and aliquots were taken during the process. After the reaction was done, the solution was cooled naturally to room temperature, and the nanoparticles were separated by adding acetone to precipitate and centrifuging at 3300 rpm for 2 min, then re-dispersed in hexanes for further analysis.

CdTe with first absorption peak at 500 nm. The asymmetric CdTe nanostructures with first absorption at 460 nm were synthesized using an injection method. Typically, 0.10 mmol CdAC₂·2H₂O, 0.20 mmol selected chiral ligand, and 4.00 g ODE were charged in a three neck-flask. The flask was connected to a schlenk line and pumped at room temperature for 3 times to remove oxygen and water. Then the reaction was heated to 180° C. under argon flow, and 100 μL of 1 M TOP-Te was injected into the flask. The reaction was maintained at 180° C. for 45 min under argon flow, and aliquots were taken during the process. After the reaction was done, the solution was cooled naturally to room temperature, and the nanoparticles was separated by adding acetone to precipitate and centrifuge at 3300 rpm for 2 min, then re-dispersed in hexane for further usage.

### Characterization

UV-Vis absorbance spectroscopy. UV-Vis absorbance spectra were measured by Shimazu UV-1800 operating from 200 nm to 800 nm. Liquid samples were measured with a 1.0 cm optical path length cuvette. The absorbance of the major peak was adjusted to 0.20 to 0.80 by controlling the concentration of sample for optimized UV signals.

Circular dichroism spectroscopy. Circular dichroism (CD) spectra were measured by Applied Photophysics Chirascan™ with xenon lamps operating from 200 nm to 800 nm. Liquid samples were measured in a 1.0 mm optical path length cuvette to minimize the solvent background signal. The absorbance of the major peak was adjusted to 0.2 to 0.8 by controlling the concentration of sample for optimized CD signals.

Transmission electron microscopy (TEM). TEM, high resolution-TEM (HRTEM), and scanning-TEM (STEM) images were measured by FEI F20 S/TEM operating at 80 KV and 200 kV under room temperature. TEM tomography data were measured by ThermoFisher Scientific Titan Krios Cryo-TEM equipped with double tilt holder operating at 300 KV under cryo-temperature.

Nanostructure samples were first dispersed in hexane to form a uniform solution, then the solution was directly dropped onto an ultrathin carbon support film of approximately 3 nm in thickness (Ted Pella) to achieve optimized contrast.

Vibrational circular dichroism (VCD). VCD from 1500 cm⁻¹ to 900 cm⁻¹ was measured by BioTools CHIRALIR-2X equipped with 1 mm optical path length cuvette made of CaF₂ crystal. The absorbance of the highest peak was adjusted to 0.6-1.0 by controlling the concentration of sample, and the measurement for each sample was repeated 4000 to 8000 times for optimized VCD signals.

### Circular Dichroism Theory and Calculations

Circular dichroism spectroscopy is one of the most widely applied methods for characterizing asymmetric nanostructures and was used throughout the study in this chapter. In this section, some essential equations of circular dichroism spectrum were derived and discussed.

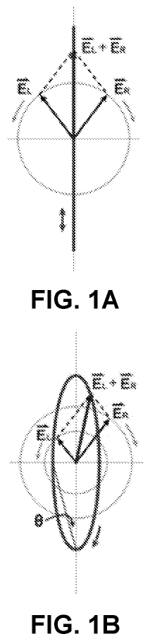

Circular dichroism (CD) spectroscopy uses an oscillated left-hand and right-hand circularly polarized light as incident beam and measures the absorbance difference of a specific sample for left-hand and right-hand circularly polarized light. Generally, a pair of left-hand and right-hand circular polarized light with equal intensity combines into linearly polarized light, while a pair of left-hand and right-hand circular polarized light with different intensity combines into elliptically polarized light (ellipticity of θ). As shown in FIGS. 1A-1B, E_{L} and E_{R} are the amplitude of the left-hand and right-hand circular polarized light, respectively. During CD spectroscopy measurement, a beam of linear polarized light shines through the sample and the ellipticity of the transmitted beam is measured. Since the sample have different molar absorbance for left-hand and right-hand circular polarized light (ε_{L} and ε_{R} in unit of cm⁻¹M⁻¹), the transmitted beam is elliptically polarized and composed of left-hand and right-hand circular polarized light at different intensity (I_{L} and I_{R}). Considering the intensity of left-hand and right hand circular polarized light for incident beam is identical (I₀), with Lambert-Beer's Law, the intensity of transmitted left-hand and right-hand circularly polarized light is calculated as follows:

$${I}_{L} = {I}_{0} \cdot {10}^{- {A}_{L}} = {I}_{0} \cdot {10}^{- {ε}_{L} cl} = {I}_{0} \cdot {e}^{- ( {ε}_{L} cl ) ln 10} \qquad ( 1 )$$

$${I}_{R} = {I}_{0} \cdot {10}^{- {A}_{R}} = {I}_{0} \cdot {10}^{- {ε}_{R} cl} = {I}_{0} \cdot {e}^{- ( {ε}_{R} cl ) ln 10} \qquad ( 2 )$$

where A_{L} and A_{R} are the absorbance of left-hand and right-hand circular polarized light, respectively, c is the molar concentration (M), l is the optical path length (cm). As depicted in FIGS. 1A-1B, the ellipticity is calculated as follows,

$$tan θ = \frac{{E}_{R} - {E}_{L}}{{E}_{R} + {E}_{L}} = \frac{{I}_{R}^{1 / 2} - {I}_{L}^{1 / 2}}{{I}_{R}^{1 / 2} + {I}_{L}^{1 / 2}} \qquad ( 3 )$$

From Equation 3, it is known that when θ=0, the light is linearly polarized, when θ=π/4, the light is right-hand circularly polarized, and when θ=π/4, the light is left-hand circularly polarized. Combining Equation 1, 2, and 3, it is derived,

$$tan θ = \frac{{e}^{- ( {ε}_{R} cl ) ln 10} - {e}^{- ( {ε}_{L} cl ) ln 10}}{{e}^{- ( {ε}_{R} cl ) ln 10} + {e}^{- ( {ε}_{L} cl ) ln 10}} = \frac{{e}^{( Δε cl ) ln 10} - 1}{{e}^{( Δε cl ) ln 10} + 1} \qquad ( 4 )$$

where Δε=ε_{L}−ε_{R}.

During measurements, the overall absorbance of a sample should be adjusted to between 0.2 to 0.8 for the optimized signal to noise ratio (S/N). Because the usual absorbance difference of left-hand and right-hand polarized light is extremely small (in the order of 10⁻⁵ to 10⁻³ of total absorbance), Δεcl<<1, and θ→0, tan θ≈θ. Furthermore, Equation 5 could be approximated by first order Taylor expansion for the exponentials,

$$θ ( radians ) = Δε cl ( \frac{ln 10}{4} ) = 0.9 \; 163 \cdot Δε cl = 0.9 \; 163 \cdot Δ A \qquad ( 5 )$$

$$or θ ( degree ) = Δε cl ( \frac{ln 10}{4} ) ( \frac{180}{π} ) = 32.9 \; 82 \cdot Δε cl = 32.9 \; 82 \cdot Δ A \qquad ( 6 )$$

where ΔA=A_{L}−A_{R}, is the absorbance difference of the left-hand and right-hand circularly polarized light.

Typically, the result of CD characterization is expressed as the ellipticity at difference. Occasionally, the absorption of left-hand and right-hand circularly polarized light is also essential. Thus, based on ellipticity and total absorbance, the calculation of the absorbance for left-hand and right-hand circularly polarized light (A_{L} and A_{R}, respectively) is discussed in the following.

From the CD characterization, the ellipticity and total absorbance are measured as θ(mdegree) and A_{T} respectively. The intensity of transmitted beam is the sum of the intensity of left-hand and right-hand polarized light. Therefore, based on Lambert-Beer's Law, the total absorbance (A_{T}) is calculated as:

$${A}_{T} = - log \frac{{I}_{T}}{{I}_{T , 0}} = - log \frac{{I}_{L} + {I}_{R}}{{I}_{0} + {I}_{0}} = - log \frac{{I}_{L} + {I}_{R}}{2 {I}_{0}} \qquad ( 7 )$$

where I_{T,0}=2I₀. Combing Equation 1, 2 and 7,

$$1 {0}^{- {A}_{T}} = ( 1 {0}^{- {A}_{L}} + 1 {0}^{- {A}_{R}} ) / 2 \qquad ( 8 )$$

Since ΔA=A_{L}−Δ_{R}, Equation 8 is rewritten as two different formats,

$$2 \cdot {10}^{- {A}_{T}} = 1 {0}^{- Δ A - {A}_{R}} + 1 {0}^{- {A}_{R}} \qquad ( 9 )$$

$$2 \cdot {10}^{- {A}_{T}} = 1 {0}^{- {A}_{L}} + 1 {0}^{- {A}_{L} + Δ A} \qquad ( 10 )$$

Solving Equation 9 and 10,

$${A}_{R} = {A}_{T} + log ( 1 {0}^{- Δ A} + 1 ) - log 2 \qquad ( 11 )$$

$${A}_{L} = {A}_{T} + log ( 1 {0}^{Δ A} + 1 ) - log 2 \qquad ( 12 )$$

Combing Equation 6 with 11 and 12,

$${A}_{R} = {A}_{T} + log ( 1 {0}^{- θ / 32.982} + 1 ) - log 2 \qquad ( 13 )$$

$${A}_{L} = {A}_{T} + log ( 1 {0}^{θ / 32.982} + 1 ) - log 2 \qquad ( 14 )$$

where A_{T} is obtained from absorption spectrum, and θ in degrees is extracted from CD spectrum. Both are the results from CD characterization.

### Example 2: Results and Discussion

### CdSe Chiral Nanostructures

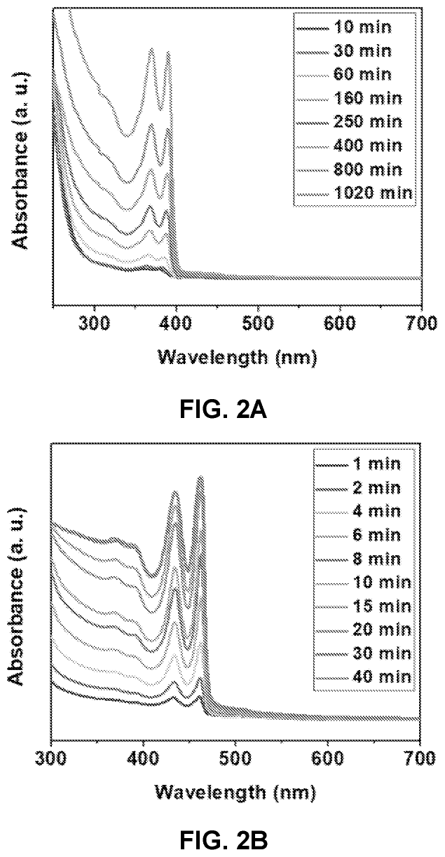

The growth of nanostructures. FIGS. 2A-2B shows the overlaid UV spectra of the aliquots of CdSe nanostructures synthesized with C10Cho, under different reaction conditions. Two different types of nanostructures were noticed: CdSe-390 with the first absorption peak at 390 nm (a), and CdSe-460 with the first absorption peak at 460 nm (b). Both reactions did not present any peak shift, suggesting that their thicknesses were well controlled and maintained during the synthesis. Based on reported literature, such phenomena indicated that 2D nanostructures were obtained and the thicknesses of these two types of nanostructures was calculated to be 1.22 nm for CdSe-390 and 1.83 nm for CdSe-460.

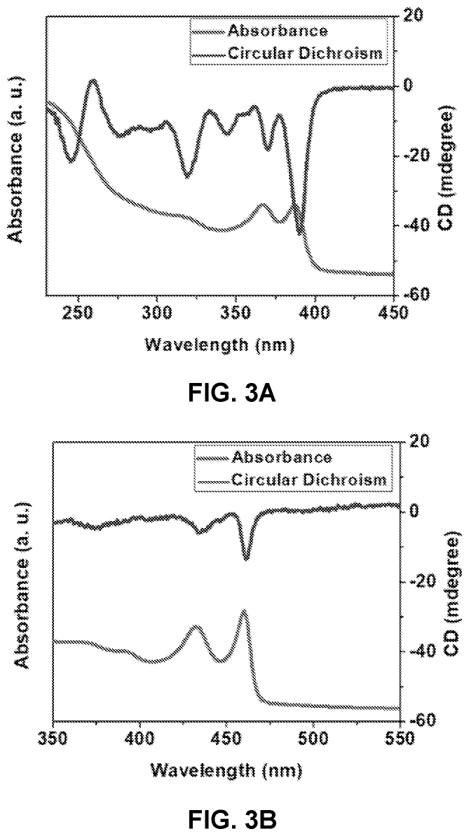

CD spectrum. FIGS. 3A-3B shows the CD spectrum of CdSe-390 (a) and CdSe-460 (b). For CdSe-390, two major CD bands at the position of the two major absorption bands were found at 390 nm and 370 nm, associated with heavy-hole and light-hole electronic transition, which suggested that the intrinsic chirality of nanostructure. Besides these two bands, extra CD bands were found at 343 nm, 320 nm, 275 nm and 245 nm. These bands were related to the electronic transitions of the chiral nanostructure at higher energy level. For CdSe-460, only two CD bands were found at 460 nm and 430 nm which corresponding to the major absorption bands for heavy-hole and light-hole electronic transitions. It should be mentioned that all these CD bands were right-handed, which was identical to the direction of chirality for cholesterol molecule, indicating that, with identical chiral center in cholesterol molecule, the thickness of nanostructure will not affect the direction of chirality. However, compared with CdSe-460, CdSe-390 exhibited about 4 times stronger CD signal at the first absorption peak. Moreover, more peaks of higher energy electronic transition bands were resolved for CdSe-390 than for CdSe-460. Therefore, it is concluded that although thickness did not result in any change in the direction of chirality, thinner 2D nanostructure generated stronger chirality than thicker nanostructure did.

### CdS Chiral Nanoparticles

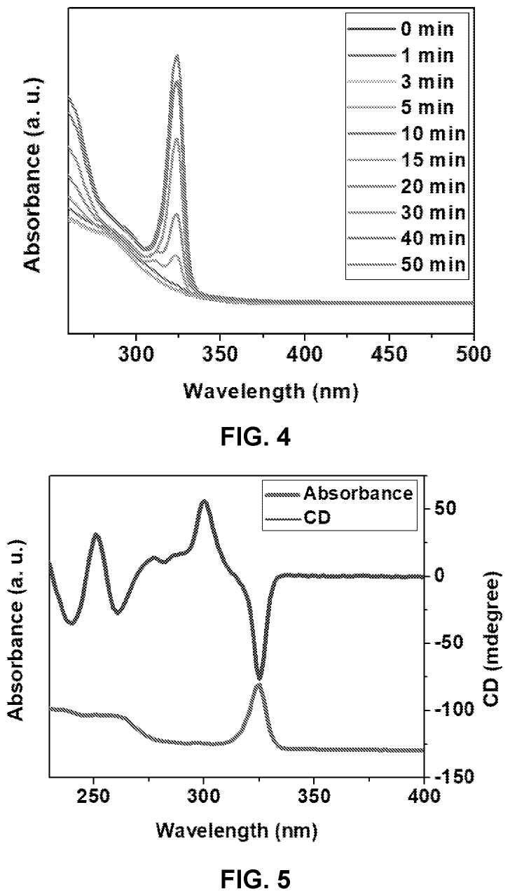

The growth of nanostructures. Based on the success of CdSe chiral nanostructure synthesis, the thinnest reported CdS 2D nanostructure with chiral ligand are prepared and studied, with its first absorption peak at 324 nm. FIG. 4 shows the overlaid UV spectra of aliquots of CdS-324 synthesized with C10Cho. During the first 10 minutes of reaction, very tiny atomic clusters were generated which were not detectable under UV-Vis spectrum. After 10 minutes, the critical concentration limiting supersaturation point was reached, and the nucleation started. The aliquots at 15 min and 20 min had two peaks at 310 nm and 324 nm, corresponding to the CdS cluster monomer and 2D CdS nanostructure, respectively. Based on literature reporting, the thickness of CdS-324 nanostructure was calculated to be 1.22 nm.

CD spectrum. FIG. 5 showed the UV-Vis and CD spectra of the purified CdS-324 nanostructure. At 324 nm which corresponds to the heavy-hole to electron band transition, a CD peak was identified as a right-handed preferred transition. At lower wavelength region, multiple CD peaks were noticed, including left-handed CD peaks at 300 nm and 251 nm as well as right-handed CD peaks at 261 nm and 241 nm. These results suggested that electron band structure of higher energy level shows different chirality, which requires further investigation by theoretical calculation.

### CdTe Chiral Nanostructures

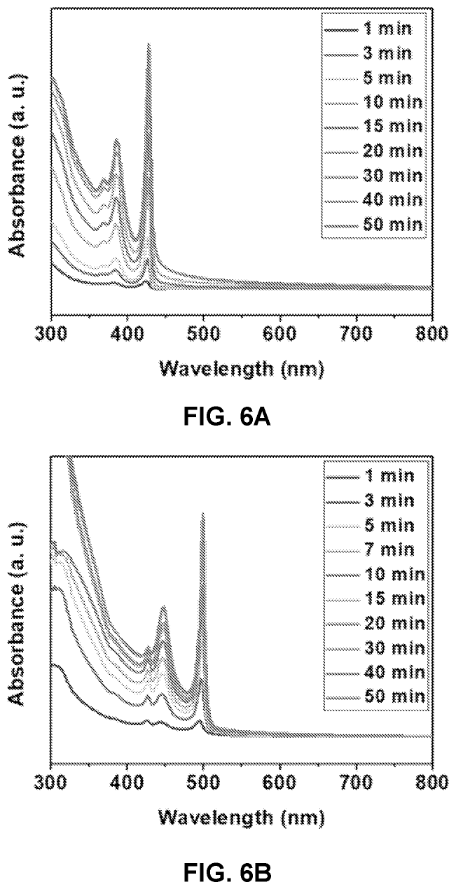

The growth of nanostructures. FIGS. 6A-6B shows the overlaid UV spectra of aliquots taken during the growth of CdTe nanostructures synthesized with C₁₀Cho. The thickness of CdTe nanostructure could be controlled by the reaction temperature. At 120° C., the nanostructure presented an absorption peak at 425 nm (CdTe-425), while at 180° C., the nanostructure presented an absorption peak at 500 nm (CdTe-500). Furthermore, prolonged reaction time only led to increased absorbance intensity but not the peak position, implying that the thickness of nanostructures was precisely controlled in a uniform fashion. The thicknesses of the two different nanostructures were calculated as 1.62 nm and 1.94 nm.

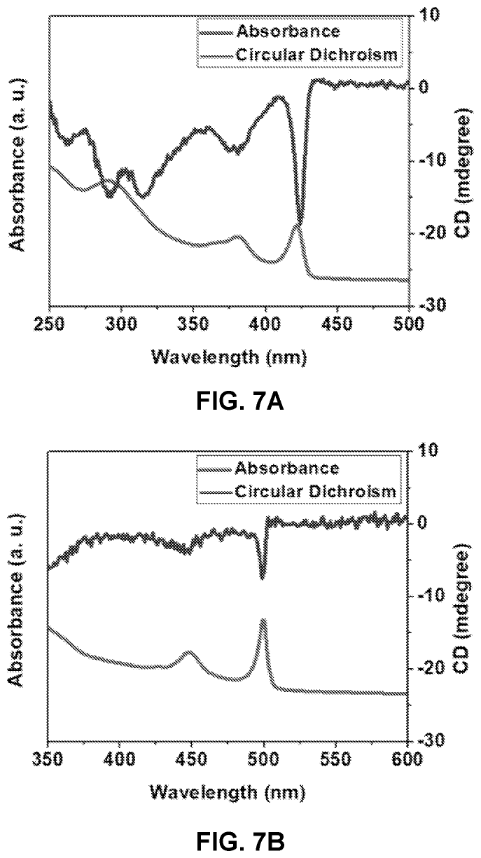

CD spectrum. CDs measurements were performed on both CdTe-425 (FIG. 7A) and CdTe-500 (FIG. 7B). For CdTe-425 chiral nanostructure, two major CD peaks at 425 nm and 380 nm were found at the positions of the two major absorption bands associated to heavy-hole and light-hole electronic transitions, indicating that nanostructure itself has chirality. Besides these two bands, at 314 nm and 290 nm, CD bands corresponding to higher energy electronic transitions were found. For CdTe-500 chiral nanostructure, only two CD bands were identified at 500 nm and 450 nm, which corresponds to the major absorption bands for heavy-hole and light-hole electronic transitions.

Similar to CdSe chiral nanostructure, all these CD bands were right-handed, which was identical to the direction of chirality for cholesterol molecule, suggesting that the thickness did not affect the direction of chirality for nanostructure synthesized with ligands of identical chiral center. Compared with CdSe-500, CdTe-425 showed about 3 times stronger CD signal at the first absorption peak. While for higher energy electronic transition bands, more peaks were resolved from CdTe-425 chiral nanostructure, indicating that although thickness did not change the direction of chirality, thinner 2D nanostructures generated stronger chirality than thicker ones.

Nanostructure Synthesized with Pseudo-Enantiomer

With successful synthesis of three types of chiral nanostructures (CdSe, CdS, and CdTe) in non-polar organic phase, chirality averting was evaluated and tested by applying the enantiomer of C₁₀Cho ligand during nanostructure synthesis, since the chirality of nanostructure was speculated to come from the template effect of chiral ligands.

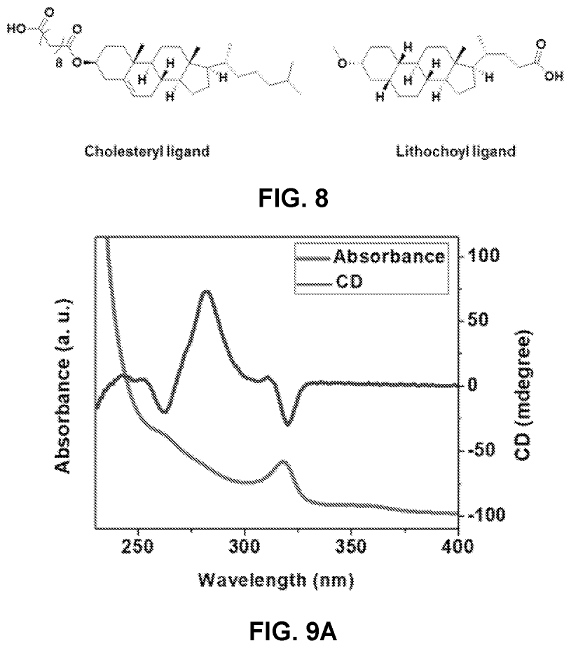

Because of the time and cost of total synthesis of cholesterol enantiomer, a pseudo-enantiomer ligand-lithocholic acid methyl ether (FIG. 8) was designed and used. Compared with cholesterol-based ligand, the pseudo-enantiomer ligand possessed a similar chiral ring, therefore was expected to have nearly identical template effects during nanoparticle synthesis. However, the carboxylic acid group of lithocholic acid methyl ether is on the 5-member ring side of the chiral center, which should give an opposite chirality comparing to cholesterol-based ligand.

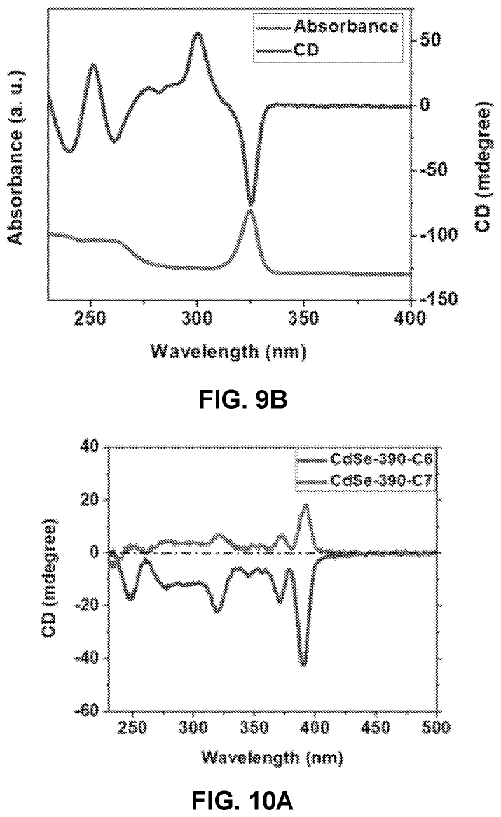

With this pseudo-enantiomer ligand, CdS chiral nanostructures were synthesized. As shown in FIGS. 9A-9B, with the similar CD spectrum to that of C₁₀Cho ligand sample, it was concluded that the pseudo-enantiomer ligand did not result in any chirality change for the obtained CdS nanostructures. One possible explanation for such a phenomenon was attributed to the formation of chiral nanostructure. The chirality of nanostructure arises from strong ring-ring interactions among chiral ligands, or layer by layer stacking of their ring structures in a helical pattern, specifically. As the nanostructures are growing along the template of the ring structures, the chirality of them are expected to be identical to that of the ligand template. Since the cholesterol and pseudo-enantiomer ligand share the same ring structure, they should present identical ligand template effects towards the growth of nanostructures, thus resulting in same chirality.

### Even-Odd Effect

From previous results, it was found that for CdSe, CdS, and CdTe, 2D structures, ones with smaller thickness gave stronger CD signal. Considering higher CD signal and better signal to noise ratio for CD spectrum, in this section, only the thinnest 2D nanostructures were selected for further studies.

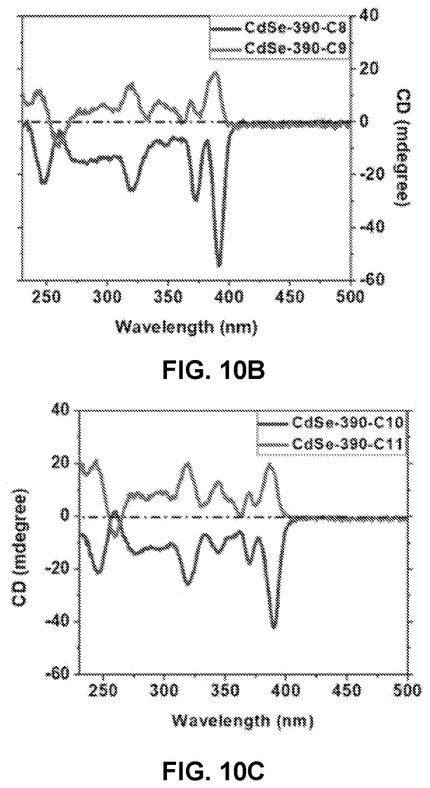

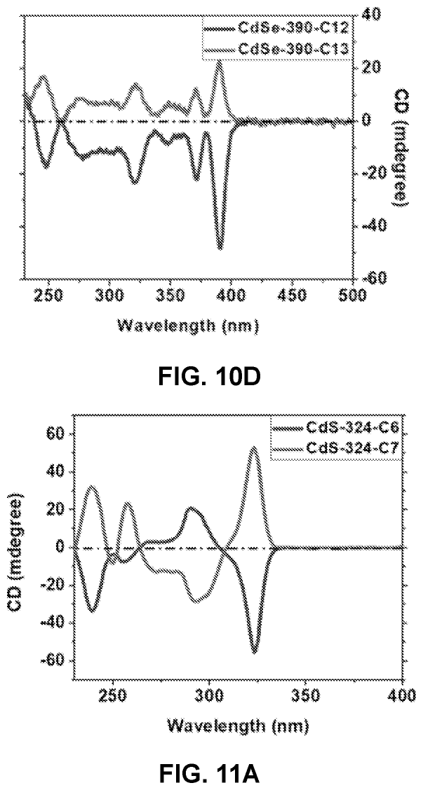

CdSe chiral nanoparticles synthesized with different cholesteryl ligands. Cholesterol-based chiral ligands with varied length of hydrocarbon chain were synthesized by using different dicarboxylic acid as reactants, then CdSe nanostructures were prepared using a series of cholesterol-based chiral ligands, from C₆Cho to C₁₃Cho. FIGS. 10A-10D show the CD spectrum of the series of CdSe-390 chiral nanostructure. From CD spectrum, all the types of nanostructures possessed almost identical CD band position and peak width. Surprisingly, the chirality varied among nanostructures with varied ligands of different carbon chain length. Moreover, the direction of chirality alternated as the hydrocarbon chain length increased. Specifically, all the nanostructures synthesized with hydrocarbon chain length in even numbers, 6, 8, 10, and 12, showed right-handed chirality, while the nanostructures synthesized with hydrocarbon chain length in odd numbers, 7, 9, 11, and 13, showed left-handed chirality. Each CD transition peak perfectly mirrored as chirality alternated without any peak shift. With these results, it was concluded that all the prepared nanostructures shared the same chiral structure. However, ligands of the same chiral center but even or odd carbon chain length resulted in nanostructures mirroring each other. Such phenomenon is called “Even-Odd” effect.

To confirm that the ester formation reaction did not alternate the chirality of cholesterol-based ligands, optical rotation and CD spectrum were measured for a pair of even and odd ligands, C₉Cho and C₁₀Cho. From the optical rotation characterization, the specific rotation of C₉Cho was −24.10±0.05 deg dm⁻¹ mL g⁻¹, and for C₁₀Cho was −24.61±0.09 deg dm⁻¹ mL g⁻¹. Considering that the specific rotation for cholesterol is −31.5 deg dm⁻¹ mL g⁻¹, it was concluded that the chirality of C₉Cho and C₁₀Cho was nearly identical, and the ester formation reaction did not alternate the chirality of cholesteryl ring structure.

From previously published literature, the chirality of nanostructures was either controlled by the chirality of ligands (e.g., using a pair of enantiomer ligands), or applying different reaction conditions to induce chiral assembly (e.g., using circular polarized light to introduce asymmetry in the reaction system). However, our results revealed that using a pair of ligands with the same chirality, nanostructures in opposing chirality were achieved, which, to the best of our knowledge, has never been reported before.

One possible explanation for such phenomena could be addressed to the close-packed ligands capping the nanostructures. Because of such close-packing, the hydrocarbon chains of the ligands are no longer flexible as free ligand molecules in solution. Instead, rigid zig-zag structures were preferred. Therefore, the end carboxylic acid groups anchored on nanostructures' surface are forced to point at different directions as the length of carbon chain alternates between even and odd, which results in nanostructures with opposing chirality.

CdS and CdTe chiral nanoparticles synthesized with different cholesteryl ligands. With a confirmed Even-Odd effect on CdSe-390 nanostructures, CdS-324 and CdTe-420 chiral nanostructures were also synthesized using cholesteryl ligands with different hydrocarbon chain lengths and characterized.

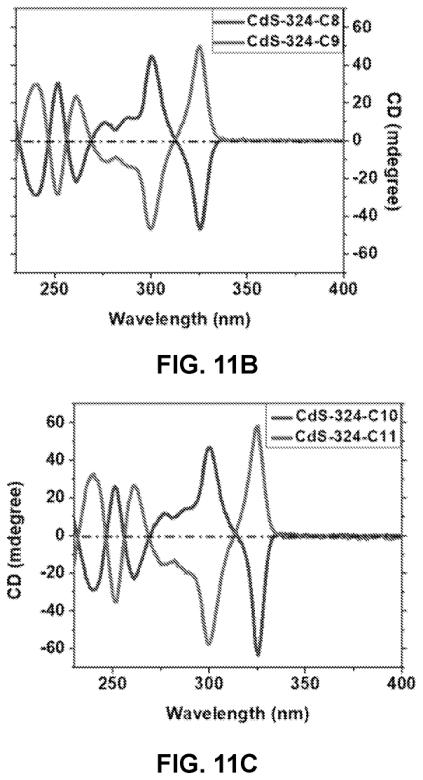

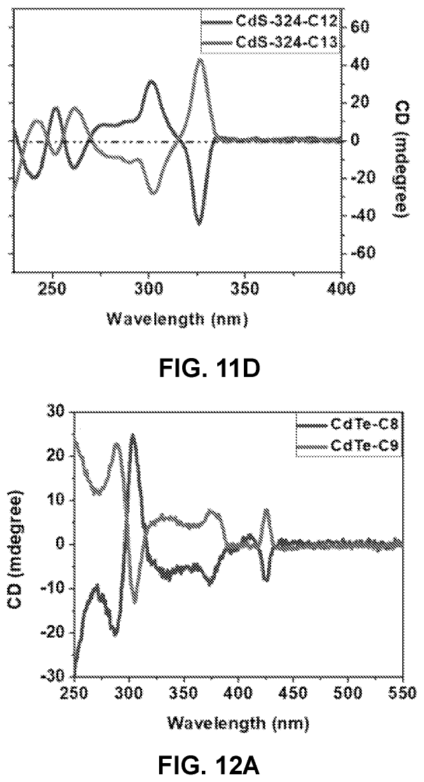

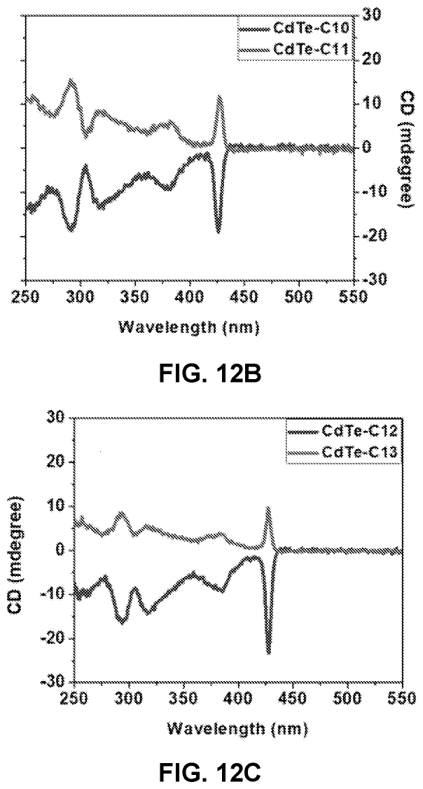

FIGS. 11A-12C showed the CD spectra for CdS-324 and CdTe-420 chiral nanostructures synthesized with cholesteryl ligands of even/odd numbers in carbon chain length, respectively. Obviously, the Even-Odd effect was also observed on CdS and CdTe 2D nanostructures.

### Vibrational Circular Dichroism

Vibrational Circular Dichroism (VCD) is a type of characterization technology for measuring chiral molecules' absolute configurations. Recently it has been applied on determining the chiral structures of macromolecules and large structures. Similar to UV-Vis CD, which measures the absorption difference of a pair of circularly polarized light in UV-vis range, VCD measures the absorption difference of a pair of circularly polarized light in infrared range. VCD is also utilized for measuring the molecule-molecule interactions under asymmetric field.

For better understanding of the ligand-stacking and nanostructure charity, VCD measurements were carried out for CdSe nanostructures synthesized with C₉Cho and C₁₀Cho ligands.

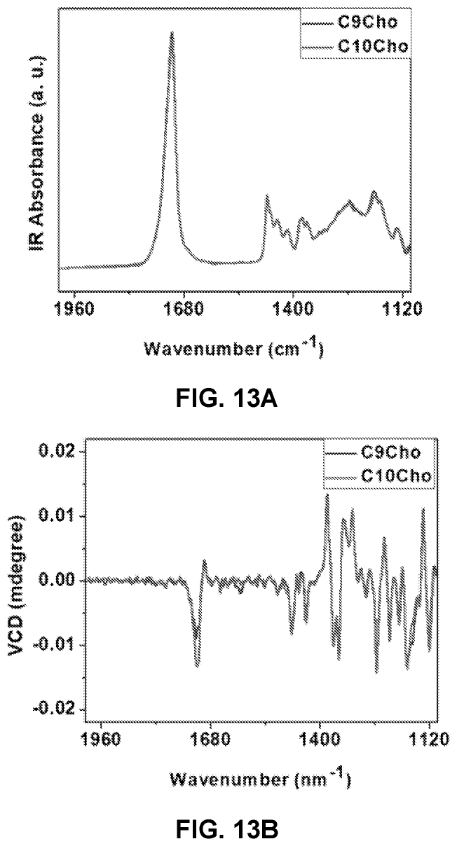

Firstly, the VCD spectrum of pure cholesteryl ligands were measured to confirm that the modification of cholesterol did not alternate its chirality. As shown in FIGS. 13A-13B, for both types of ligand molecules, the IR and VCD spectrum aligned perfectly, indicating that the ester forming reaction did not alternate the chirality of cholesterol. In IR spectrum, at 1710 cm-1, a clear strong absorbance band associated to the C═O stretch mode of the ester bond and the carboxyl group was observed. At the same wavenumber, a strong VCD peak was also observed with same chirality for both ligands, indicating that different carbon chain length did not alternate the chirality of the ligand molecules. Below 1500 cm⁻¹ were the absorbance bands of cholesterol backbone, in this area the IR and VCD spectrum were nearly identical, further confirmed that both ligands possessed same chirality. Comparing to the optical rotation of C₉Cho and C₁₀Cho was also measured as −24.095 deg dm⁻¹ mL g⁻¹ and −24.608 deg dm⁻¹ mL g⁻¹ respectively, further proving that both ligands had identical chirality.

After confirming the ligands' identical chirality, VCD of CdSe nanostructure with different cholesteryl ligands were measured. Previous CD measurements in UV and visible wavelength offered information of nanostructures' asymmetric electronic band structure, while the VCD measurement provided insights of the asymmetric organic functional group structure. Specifically, the Cd atoms are directly bonded to the carboxylic acid groups of the ligands. By measuring the carboxyl group on the surface of the nanostructure should give a better idea of the chirality of the inorganic core. Thus, with VCD measurement, the overall relationship of chiral ligands on the nanostructures' surface was explored.

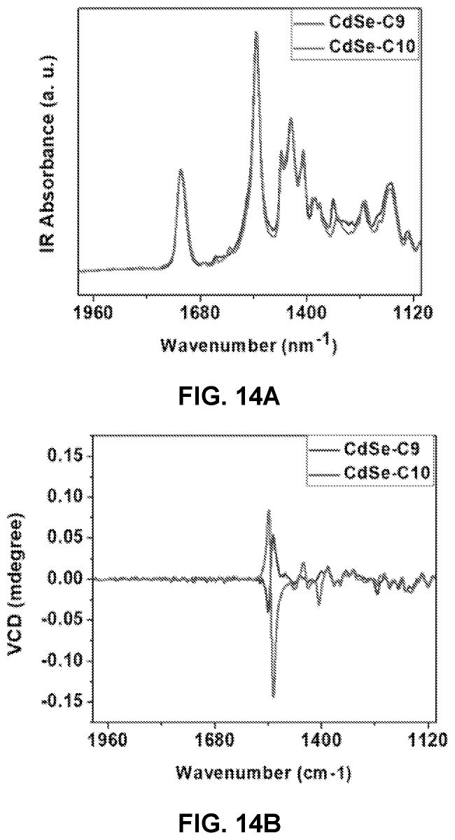

FIGS. 14A-14B show the IR and VCD spectrum of the CdSe chiral nanostructure synthesized with C₉Cho and C₁₀Cho. Due to the formation of cadmium carboxylate, the vibrational band of C═O stretch in the carboxyl group shifted from 1710 cm⁻¹ to 1533 cm⁻¹. Specifically, the VCD showed a duplex peak for CdSe synthesized by C₉Cho and C₁₀Cho at 1533 cm⁻¹ and the duplex peak was mirrored to each other, indicating that while the vibrational coupling between the surface carboxyl group were identical, the chirality was reversed from C₉Cho to C₁₀Cho. Another major difference between these two samples in VCD spectrum appeared at the peak of 1406 cm⁻¹ which was also possibly addressed to the asymmetric stretching of cadmium carboxylate. It also should be noted that the overall VCD intensity of CdSe—C9 was weaker than that of CdSe—C10, with the ratio of which proportional to the CD signal strength measured with UV-Vis light. With these results, it was concluded that the surface structure of chiral CdSe synthesized with C₉Cho and C₁₀Cho were a pair of enantiomers.

### TEM Characterization and Model Construction

Obviously the most direct methods to characterize the overall shape of nanostructure are imaging techniques. Among them, TEM is one of the best tools to analysis structures in nanometer scale. Therefore, CdSe—C9 and CdSe—C10 chiral nanostructures were characterized by TEM imaging.

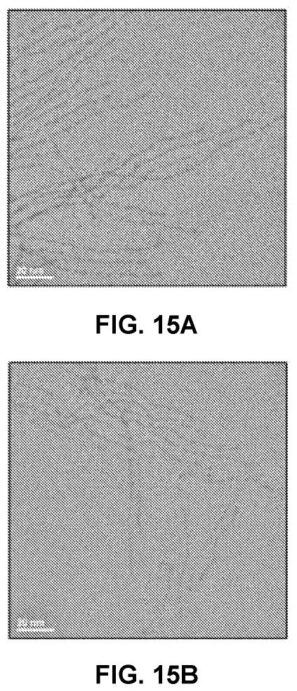

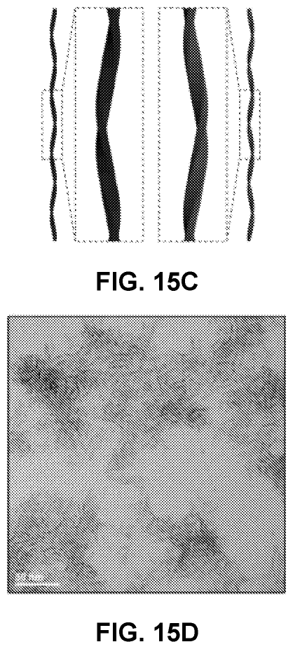

FIGS. 15A-15D show the TEM images of CdSe—C9 (FIG. 15A) and CdSe—C10 (FIG. 15B). Both CdSe—C9 and CdSe—C10 possessed a nanoribbon like structure with some “knuckles” along the long axis. The length of the overall nanostructures was about 100 nm, and the width was 1.9 nm on the thickest part (labeled with arrows) and 1.2 nm on the thinnest part (labeled with arrows). As mentioned in previous parts, the thickness of nanostructures was calculated to be 1.22 nm. Considering that the TEM image is a 2D projection of a 3D object, based on current imaging data, it was predicted that the overall shape of these two different types CdSe nanoribbon was a pair of helix, one being left-handed while the other being right-handed. The thicker part along the ribbon was the width of the helix, while the thinner part was the thickness of the ribbon projected on the camera. FIG. 15C shows a predicted 3D model of a helical nanoribbon and its corresponding 2D projection. Based on this model, the pitch of the helical structure was calculated as 18 nm. This is two times the length between each “knuckles” on the TEM images. As a comparison, FIG. 15D showed the TEM image of CdSe nanostructure synthesized with non-chiral ligand, myristic acid, which gave no CD signal. For such achiral CdSe nanostructure, a flat 2D nanoribbon structure was observed in image. Therefore, the twisting along the nanostructure is essential for its chiral optical activity.

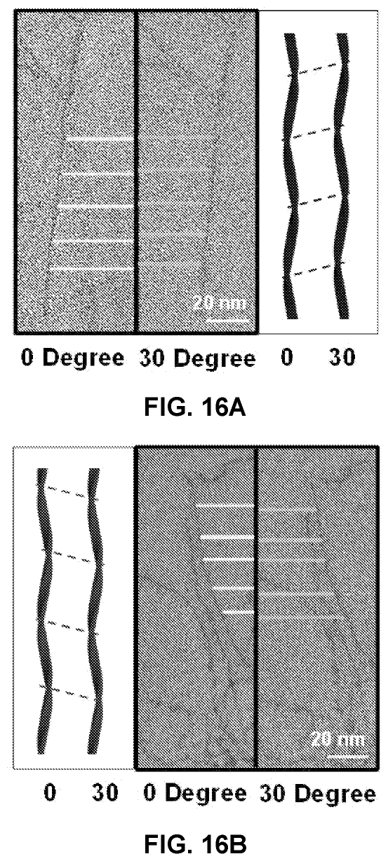

To characterize the rotation direction of the ribbon, CdSe—C9 and CdSe—C10 were tilted under TEM imaging. Because of the helical structure, the thicker part and thinner part should migrate along the ribbon as shown in FIGS. 16A-16B with 3D model. Therefore, 30-degree tilt at the same direction was applied on CdSe—C9 and CdSe—C10. From FIGS. 16A-16B, it can be found that after 30-degree tilt, the thicker and thinner part of CdSe—C9 migrated UP for 3.0 nm, and for CdSe—C10, migrated DOWN for 2.9 nm. This is solid evidence that the pair of CdSe nanoribbon is mirrored to each other. Also, the pitch for the CdSe—C9 and CdSe C10 nanoribbon could be calculated as 18.0 nm and 17.4 nm, respectively, and CdSe—C9 shows a left handed helical structure, while CdSe—C10 shows a right handed helical structure.

### Alternative Ring-Ring Interaction of Cholesteryl Ligands

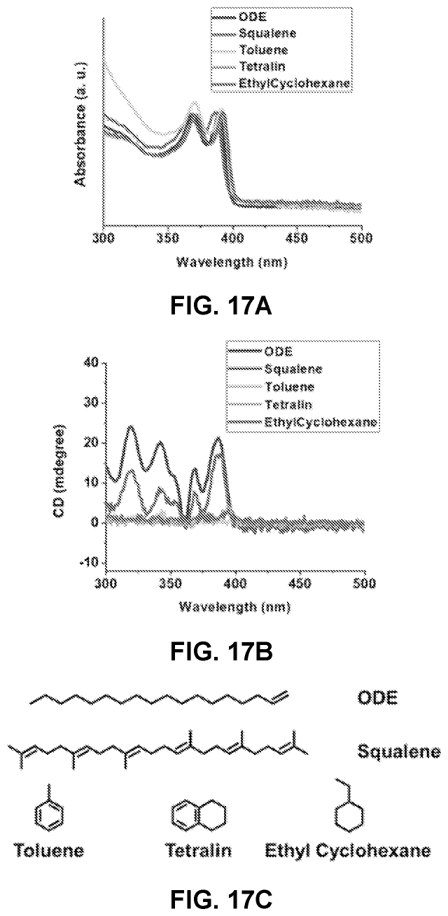

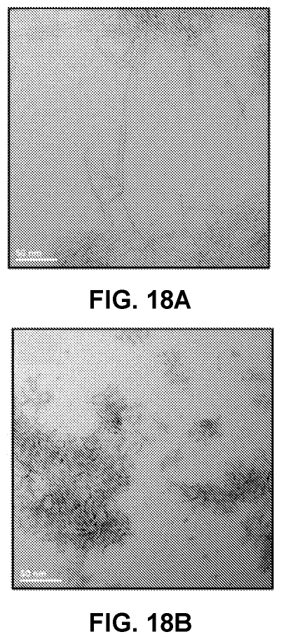

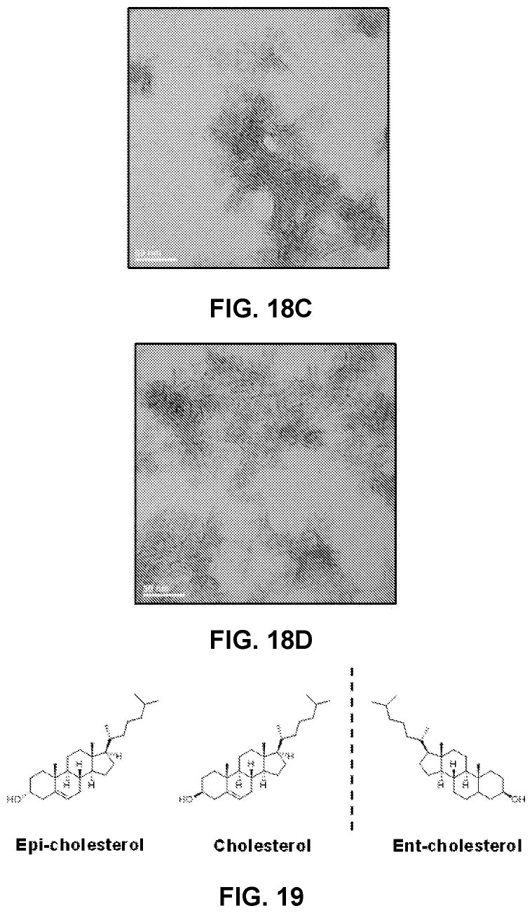

The chirality of CdSe nanostructure originates from the template effect of cholesterol ligand backbone, thus should be influenced by perturbating the cholesteryl ring-ring interaction. Therefore, for the purpose of adjusting ring-ring stacking, non-cholesteryl ring-containing guest molecules were introduced during nanostructure synthesis. FIGS. 17A-17C show the UV-Vis and CD spectrum of CdSe—C9 synthesized with guest molecules of tetralin, ethyl cyclohexane, toluene, and squalene. Within these four guest molecules, tetralin, ethyl cyclohexane, and toluene contain 6-ring structure, while squalene is a branched hydrocarbon chain (molecular structure of the four guest molecules presented in FIG. 17C). Nanoparticles synthesized with different guest molecules presented nearly identical UV-Vis spectrum as those of their counterparts synthesized without guest molecules, indicating the general size and shape of nanostructures maintained unchanged. However, in the CD spectrum, the peak at 390 nm became nearly non-detectable for CdSe—C9 synthesized with tetralin, ethyl cyclohexane, and toluene, suggesting the loss of structural chirality. As a control, CdSe—C9 synthesized with squalene exhibits un-changed CD spectrum, indicating that the 6-member carbon ring structure is the key to suppress the ligand template. FIGS. 18A-18D showed the TEM image of CdSe—C9 synthesized with guest molecules. A relatively flat 2-D structure with un-defined edge was observed instead of helical twisted nanoribbon like structure was found in the CdSe—C9 synthesized with ring-structured guest molecule, which is in consistent with the previous model that the twisting was critical to the chiral optical activity. As a control experiment, addition of the same guest molecules into synthesized chiral nanostructures at room temperature or anneal synthesized chiral nanoparticles in such guest molecule solvents did not result in any CD signal change.

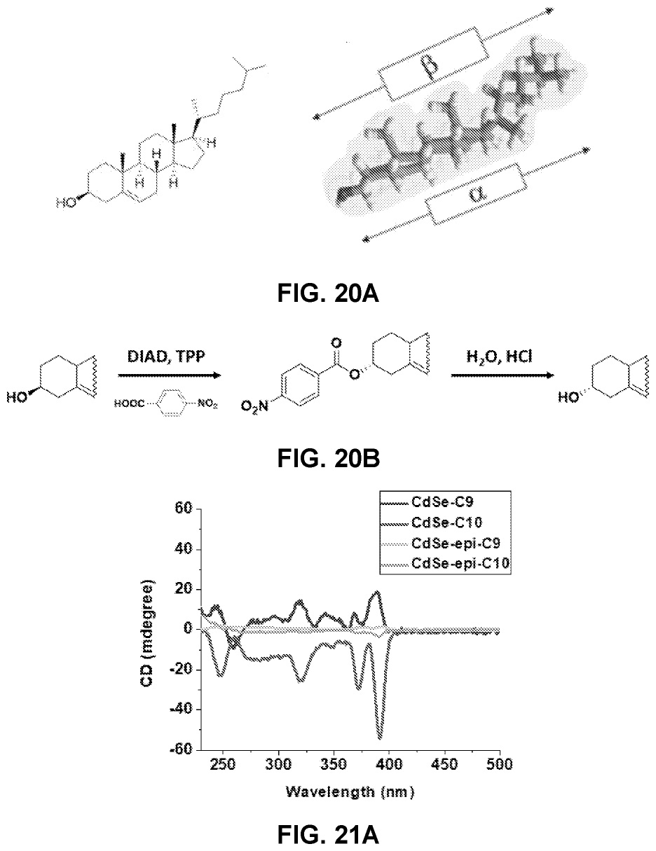

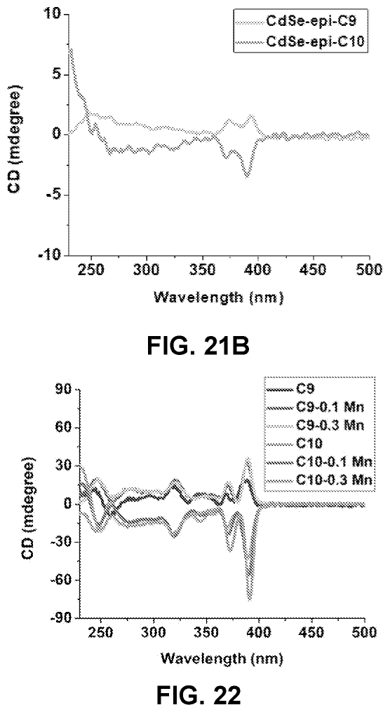

To further understand the effects of cholesterol ring-ring stacking on nanostructures' chirality, instead of using guest molecules, the ring-ring interaction was perturbed by using the diastereomer of cholesterol. It is known that cholesterol has three different types of structures, cholesterol from natural product, enantiomer of cholesterol (ent-cholesterol), and diastereomer of cholesterol (epi-cholesterol), as shown in FIG. 19. Cholesterol has two different face, a face and B face. a face is smooth among which most of the ring-ring interaction happens. B face is the rough because of the methyl group. For epi-cholesterol, the hydroxyl group interrupts the smooth alfa face, therefore, the ring-ring interaction between cholesterol is disturbed. Following this idea, epi-cholesterol was prepared using Mitsunobu reaction of cholesterol and 4-nitrobenzoic acid, deprotect, and epi-C₉Cho or epi-C₁₀Cho ligands were synthesized through regular ester formation reaction with azelaic acid (C9) or sebacic acid (C10) (FIGS. 20A-20B). FIGS. 21A-21B show the CD spectrum of CdSe synthesized with epi-ligands (CdSe-epi-C9 and CdSe-epi-C10). Compared with CdSe chiral nanostructures synthesized with regular cholesteryl ligands, both types of nanostructures synthesized with epi-ligands possessed identical electronic band structures, suggesting that epi-ligands did not result in any obvious change in general size or shape of the prepared nanostructures. However, nanostructures synthesized with epi-ligands presented significantly decreased CD signals, indicating the weakened chirality. Even with weak CD signal, the chirality still follows “Even-Odd” rule. From these results, it was concluded that with weakened ring-ring stacking among epi-ligands on nanostructures' surface, the chirality was largely decreased.

Ent-cholesterol is another cholesteryl structure and worthy of being tested, but the total synthesis of ent-cholesterol is not feasible. Besides, the results of the experiments involving ent-cholesterol could be predicted based on its structural similarity with regular cholesterol ligands. The corresponding nanostructures should exhibit strong and reversed chirality compared with those synthesized with regular cholesterol ligands.

### Doped Chiral Nanoparticles

From the twisted helical model of chiral CdSe chiral nanostructures, the lattice parameters should have significant defect in their crystal structure. It could speculate that atoms with different radius different to cadmium could significantly decrease the lattice strain which should result in higher optical activity. With this idea, Mn and Pb were selected as two candidates for doping CdSe chiral nanowires.

FIG. 22 shows the CD spectrum of Mn doped CdSe chiral nanostructure synthesized with C₉Cho (CdSe-C9-Mn) and C₁₀Cho (CdSe-C10-Mn). Compared to un-doped chiral nanostructure, the peak location and profile was not changed, suggesting that doping did not affect the asymmetric electronic band structure of chiral CdSe nanostructure. However, Mn-doped samples presented significantly higher CD signals than undoped ones, and the CD intensity increased with the doping level increased. Such difference could possibly be explained by following two reasons: (1) the probable interaction between Mn²⁺'s half-filled d orbital and the electronic band of CdSe chiral nanostructure; (2) the diameter difference between Cd²⁺ and Mn²⁺ may release the strain of the folded chiral nanostructure, thus result in lowered internal energy and more twisted structure, which leads to higher CD signal.

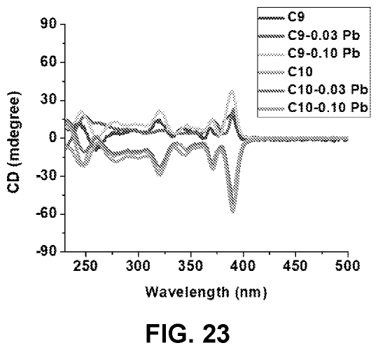

Since Pb²⁺ has the electron configuration of [Xe]6s²4f¹⁴5d¹⁰. Each orbital is fully filled, thus should not have electronic band interaction between CdSe material. FIG. 23 shows the CD spectrum of Pb—CdSe—C9 and Pb—CdSe—C10. In CD spectrum, the intensity of Pb doped chiral nanostructure is nearly identical to that of Mn doped chiral nanostructure, both being significantly higher than undoped ones. From the above results, it was concluded that the enhanced CD signal of doped chiral nanostructure was probably due to the release of lattice strain.

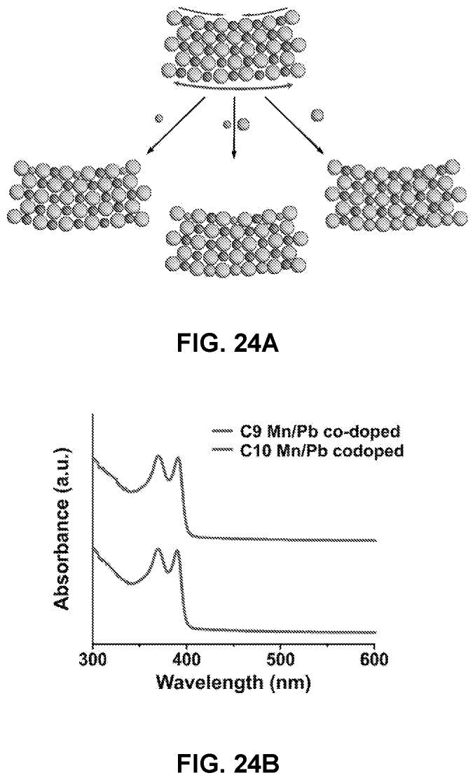

FIGS. 24A-24B illustrate the structural effects of doping with impurity elements. The chirality of the nanostructure is induced by ligand template-guided growth, resulting in a twisted nanoribbon. Due to this structural twisting, the crystal structure is bent, causing the inside face of the nanoribbon to have a smaller inter-atom distance, while the outside face possesses a larger inter-atom distance. This twisted structure inherently experiences lattice strain, which may be stabilized by the ring-ring stacking force among the ligands anchored on the nanostructures' surface. To release such lattice strain, lattice defects are often created, leading to the formation of lattice disorders in chiral nanocrystals.

In the presence of dopants, if the doped ion (Mn²⁺) has a smaller radius than Cd²⁺, it may prefer to stay on the inner curve to release certain repulsion between each ion and reduce lattice strain (FIG. 24A). Conversely, if the doped ion (Pb²⁺) has a larger radius than Cd²⁺, it may prefer to stay on the outer curve to fill in the gap caused by the bending, thereby helping to decrease lattice strain (FIG. 24A). This leads to the formation of chiral nanocrystals with fewer crystal defects and more ordered structures compared to undoped chiral nanocrystal counterparts. Therefore, improved chiral structure ordering in chiral nanocrystals results in enhanced electromagnetic field coupling with incident light, thus enhancing the CD signals of chiral nanostructures. Co-doping of Pb²⁺ and Mn²⁺ ions produces similar effects, aiding in decreasing lattice strain (FIGS. 24A-24B). It is worth noting that such co-doping allows for the synthesis of chiral nanocrystals with strong CD signals at variable levels of magnetic Mn²⁺ ion doping. These chiral nanocrystals can exhibit tunable magneto-optical CD responses, which is important for the construction of the new generation of magneto-optical devices.

### CONCLUSION

In conclusion, 2-D chiral nanostructures have been successfully synthesized in non-polar organic phase, with uniform size and shape. Also, a unique Even-Odd effect was discovered that with identical chiral center, the chirality of synthesized chiral nanostructure could be tuned by using hydrocarbon chain linker of different length. This effect was successfully confirmed on a group of II-VI two-dimensional nanostructures, including CdS, CdSe, and CdTe. The chiral CdSe nanostructure was carefully characterized using cryo-EM, and a model of chiral CdSe nanostructure was proposed and verified. Moreover, the chirality of nanostructure was found to be suppressed by using guest molecules or changing the chirality of the −OH group on the cholesterol to decrease the ring-ring interaction. Such results further prove that the cholesterol templating effect was the origin of nanostructures' chirality. Furthermore, impurity ions were successfully doped into the chiral nanostructure. Due to the difference in ionic radius, the strain of the nanostructure may be released, and the CD signal was found to be enhanced.

Further study of this project includes a deeper exploration of the electronic band structure, as some of the peaks only appeared in CD spectrum but not UV spectrum, suggesting possible chirality induced changes in electronic band structure. Additionally, the nanostructure synthesized currently does not have a high quantum yield, whereas fluorescence is a known advantage for II-VI semiconductor nanomaterials. Future studies would involve detailed study of chiral nanostructures' fluorescence properties. With the chiral II-VI nanostructures of improved fluorescence performance, such type of material could possibly pave the avenue towards another novel area of bio-imaging, that not only the wavelength, but also the chirality could be applied as imaging labels. Furthermore, impurity doped chiral nanostructures are also worthy to be explored in detail, enabling novel p-dope or n-dope chiral nanomaterials.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

### REFERENCES

1. ADAM, W. et al, ON THE MECHANISM OF THE “TRIPHENYLPHOSPHINE-AZODICARBOXYLATE (MITSUNOBU REACTION) ESTERIFICATION. Journal of the American Chemical Society 106, 1843-1845 (1984).
2. Ben-Moshe, A. et al, Chirality and chiroptical effects in inorganic nanocrystal systems with plasmon and exciton resonances. Chemical Society Reviews 42, 7028-7041 (2013).
3. Ben-Moshe, A. et al., Enantioselective control of lattice and shape chirality in inorganic nanostructures using chiral biomolecules. Nature Communications 5, (2014).
4. Che, S. et al., Synthesis and characterization of chiral mesoporous silica. Nature 429, 281-284 (2004).
5. Crassous, J. et al, “Materials for chiral light control,” Nature Reviews Materials,” (2023), 8:365-371.
6. Deubel, M. et al., Direct laser writing of three-dimensional photonic-crystal templates for telecommunications. Nature Materials 3, 444-447 (2004).
7. Dolamic, I. et al, First enantioseparation and circular dichroism spectra of Au-38 clusters protected by achiral ligands. Nature Communications 3, (2012).
8. Drezet, A. et al, Optical chirality without optical activity: How surface plasmons give a twist to light. Optics Express 16, 12559-12570 (2008).
9. Droulias, S. et al, Broad-Band Giant Circular Dichroism in Metamaterials of Twisted Chains of Metallic Nanoparticles. Journal of Physical Chemistry C 117, 1130-1135 (2013).
10. Fan, Z. Y. et al, Helical Metal Nanoparticle Assemblies with Defects: Plasmonic Chirality and Circular Dichroism. Journal of Physical Chemistry C 115, 13254-13261 (2011).
11. Fan, Z. Y. et al, Plasmonic Circular Dichroism of Chiral Metal Nanoparticle Assemblies. Nano Letters 10, 2580-2587 (2010).
12. Fantini, J. et al, How cholesterol interacts with membrane proteins: an exploration of cholesterol-binding sites including CRAC, CARC, and tilted domains. Frontiers in Physiology 4, (2013).
13. GUTHRIE, R. et al, THE MECHANISM OF THE MITSUNOBU REACTION—A P-31 NMR-STUDY. Australian Journal of Chemistry 35, 767-774 (1982).
14. Han, B. et al, Conformation Modulated Optical Activity Enhancement in Chiral Cysteine and Au Nanorod Assemblies. Journal of the American Chemical Society 136, 16104-16107 (2014).
15. Hentschel, M. et al., Optical Properties of Chiral Three-Dimensional Plasmonic Oligomers at the Onset of Charge-Transfer Plasmons. Acs Nano 6, 10355-10365 (2012).
16. Ithurria, S. et al, Continuous Transition from 3D to 1D Confinement Observed during the Formation of CdSe Nanoplatelets. Journal of the American Chemical Society 133, 3070-3077 (2011).
17. Ithurria, S. et al, Quasi 2D Colloidal CdSe Platelets with Thicknesses Controlled at the Atomic Level. Journal of the American Chemical Society 130, 16504-+ (2008).
18. Jadzinsky, P. D. et al, Structure of a thiol monolayer-protected gold nanoparticle at 1.1 angstrom resolution. Science 318, 430-433 (2007).
19. Joo, J. et al, Low-temperature solution-phase synthesis of quantum well structured CdSe nanoribbons. Journal of the American Chemical Society 128, 5632-5633 (2006).
20. JURCZAK, J. et al, INVERSION OF CONFIGURATION ON ALPHA-FURYLIC CHIRALITY CENTER—AN UNUSUAL COURSE OF MITSUNOBU REACTION. Polish Journal of Chemistry 61, 645-647 (1987).
21. Konishi, K. et al., Circularly Polarized Light Emission from Semiconductor Planar Chiral Nanostructures. Physical Review Letters 106, (2011).
22. Kumar, P. et al, “Photonically active bowtie nanoassemblies with chirality continuum,” Nature, 615, 418-424 (2023)
23. Kuzyk, A. et al., DNA-based self-assembly of chiral plasmonic nanostructures with tailored optical response. Nature 483, 311-314 (2012).
24. Lee, H. et al., Cysteine-encoded chirality evolution in plasmonic rhombic dodecahedral gold nanoparticles. Nature Communications 11, (2020).
25. Li, Z. et al, Size/Shape-Controlled Synthesis of Colloidal CdSe Quantum Disks: Ligand and Temperature Effects. Journal of the American Chemical Society 133, 6578-6586 (2011).
26. Liu, S. H. et al., Synthesis of chiral TiO2 nanofibre with electron transition-based optical activity. Nature Communications 3, (2012).
27. Liu, Y. et al, Origin of High Photoluminescence Efficiencies in CdSe Quantum Belts. Nano Letters 10, 352-357 (2010).
28. Machleidt, R. et al, Chiral effective field theory and nuclear forces. Physics Reports-Review Section of Physics Letters 503, 1-70 (2011).
29. Mahler, B. et al, Ligand-Controlled Polytypism of Thick-Shell CdSe/CdS Nanocrystals. Journal of the American Chemical Society 132, 953-959 (2010).
30. Manna, L. et al, Controlled growth of tetrapod-branched inorganic nanocrystals. Nature Materials 2, 382-385 (2003).
31. Maoz, B. M. et al., Amplification of Chiroptical Activity of Chiral Biomolecules by Surface Plasmons. Nano Letters 13, 1203-1209 (2013).
32. Mastroianni, A. J. et al, Pyramidal and Chiral Groupings of Gold Nanocrystals Assembled Using DNA Scaffolds. Journal of the American Chemical Society 131, 8455-8459 (2009).
33. Michalet, X. et al., Quantum dots for live cells, in vivo imaging, and diagnostics. Science 307, 538-544 (2005).
34. Murray, C. B. et al, SYNTHESIS AND CHARACTERIZATION OF NEARLY MONODISPERSE CDE (E=S, SE, TE) SEMICONDUCTOR NANOCRYSTALLITES. Journal of the American Chemical Society 115, 8706-8715 (1993).
35. Ouyang, J. et al., Multiple families of magic-sized CdSe nanocrystals with strong bandgap photoluminescence via noninjection one-pot syntheses. Journal of Physical Chemistry C 112, 13805-13811 (2008).
36. Pasteur, L. Sur les relations qui peuvent exister entre la forme cristalline, la composition chimique et le sens de la polarization rotatoire. Ann. Chim. Phys. 24, 18 (1848).
37. Peng, X. et al., Shape control of CdSe nanocrystals. Nature 404, 59-61 (2000).
38. Sanchez-Castillo, A. et al., On the Origin of the Optical Activity Displayed by Chiral-Ligand-Protected Metallic Nanoclusters. Journal of the American Chemical Society 132, 1504-+ (2010).
39. Schaaff, T. G. et al, Giant gold-glutathione cluster compounds: Intense optical activity in metal-based transitions. Journal of Physical Chemistry B 104, 2630-2641 (2000).
40. Seto, M. et al, Mechanical response of thin films with helical microstructures. Journal of Vacuum Science & Technology B 17, 2172-2177 (1999).
41. Shopsowitz, K. E., Free-standing mesoporous silica films with tunable chiral nematic structures. Nature 468, 422-U246 (2010).
42. Son, J. et al., Large-Scale Soft Colloidal Template Synthesis of 1.4 nm Thick CdSe Nanosheets. Angewandte Chemie-International Edition 48, 6861-6864 (2009).
43. Wang, H. T. et al., CrSi2 Hexagonal Nanowebs. Journal of the American Chemical Society 132, 15875-15877 (2010).
44. Wang, Y. et al, Emerging chirality in nanoscience. Chemical Society Reviews 42, 2930-2962 (2013).
45. Wu, X. W. et al, A novel route for synthesizing silica nanotubes with chiral mesoporous wall structures. Chemistry of Materials 19, 1577-1583 (2007).
46. Yang, Y. G. et al, Control of helical silica nanostructures using a chiral surfactant. Journal of Materials Chemistry 16, 1644-1650 (2006).
47. Yeom, J. et al., Chiral templating of self-assembling nanostructures by circularly polarized light. Nature Materials 14, 66-72 (2015).
48. Yeom, J. et al., Chiromagnetic nanoparticles and gels. Science 359, 309-+ (2018).
49. Yin, Y. et al, Colloidal nanocrystal synthesis and the organic-inorganic interface. Nature 437, 664-670 (2005).
50. Zhou, S. et al, “Chiral assemblies of pinwheel superlattices on substrates,” Nature 612, 259-265 (2022).
51. Zhou, Y. L. et al, Similar Topological Origin of Chiral Centers in Organic and Nanoscale Inorganic Structures: Effect of Stabilizer Chirality on Optical Isomerism and Growth of CdTe Nanocrystals. Journal of the American Chemical Society 132, 6006-6013 (2010).

1. A method for preparing a composition comprising a chiral nanostructure, wherein the nanostructure comprises an inorganic material, the method comprising admixing a chiral ligand and at least one inorganic-material forming precursor in a nonpolar organic solvent.

2. The method of claim 1, wherein the chiral ligand has a structure according to Formula I:
wherein A is selected from 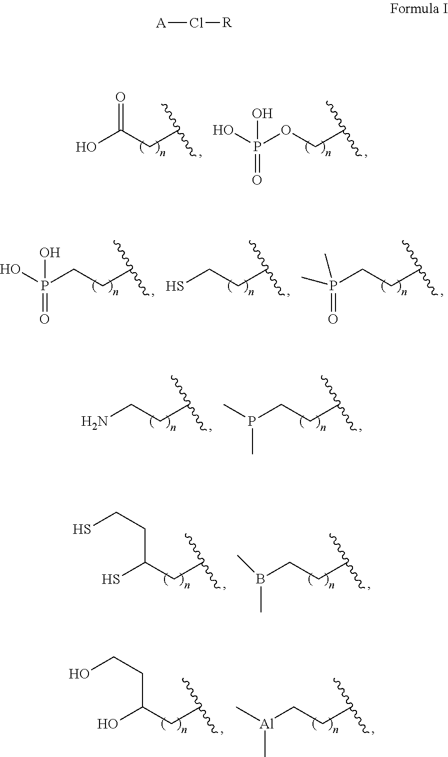
or any combination thereof
wherein n is from 1 to 14;
wherein CL is selected from 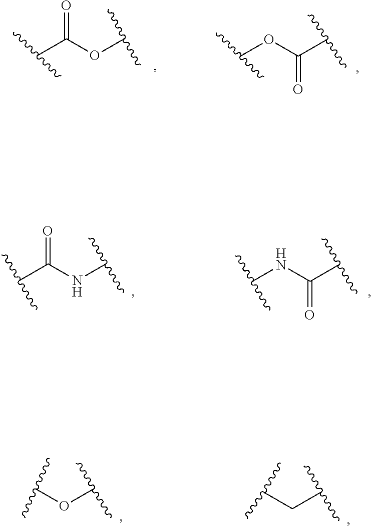
or any combination thereof; and
wherein R is selected from 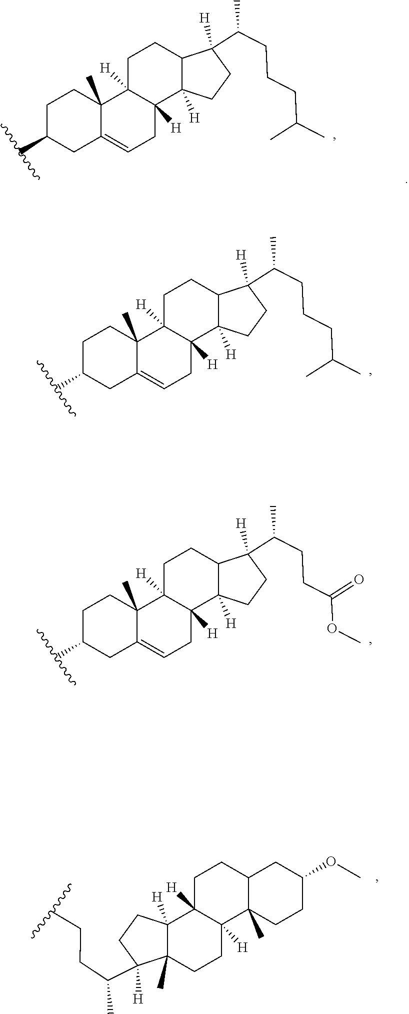 [SMILES: *[C@H]1CC[C@@]2(C)C(=CC[C@H]3[C@@H]4CC[C@H]([C@H](C)CCCC(C)C)C4CC[C@@H]32)C1 ; *[C@@H]1CC[C@@]2(C)C(=CC[C@H]3[C@@H]4CC[C@H]([C@H](C)CCCC(C)C)C4CC[C@@H]32)C1 ; *[C@@H]1CC[C@@]2(C)C(=CC[C@H]3[C@@H]4CC[C@H]([C@H](C)CCC(=O)OC)C4CC[C@@H]32)C1 ; *CC[C@@H](C)[C@H]1CC[C@H]2[C@@H]3CCC4C[C@H](OC)CC[C@]4(C)[C@H]3CC[C@]12C] 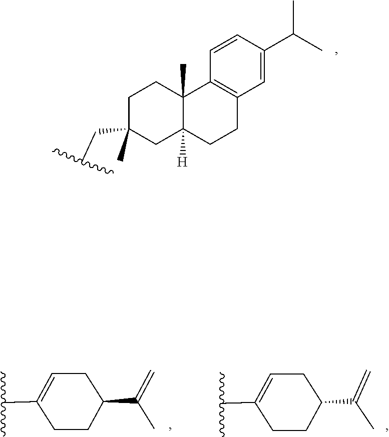
or any combination thereof.

3. The method of claim 1, wherein the chiral ligand comprises a carboxylic acid-functionalized cholesterol derivative of Formula II:

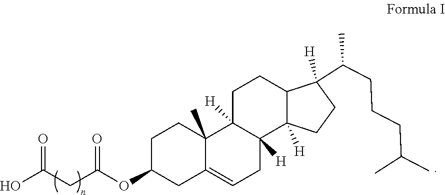

4.-5. (canceled)

6. The method of claim 1, wherein the inorganic material is a semiconductor material.

7. The method of claim 1, further comprising admixing a metal salt with the chiral ligand and the at least one inorganic-material forming precursor.

8. The method of claim 7, wherein the metal salt comprises a Cd, Cu, Pb, Hg, or Zn salt, or any combination thereof.

9. The method of claim 7, wherein the metal salt comprises a cadmium salt.

10.-11. (canceled)

12. The method of claim 1, wherein the inorganic-material forming precursor comprises a chalcogen comprising sulfur, selenium, tellurium, or any combination thereof or a chalcogen precursor comprising trioctylphosphine-Te (TOP-Te), tributylphosphine-Se (TBP-Se), or any combination thereof.

13.-15. (canceled)

16. The method of claim 1, further comprising doping the composition with a second metal, wherein the second metal comprises Pb, Mn, or any combination thereof.

17. (canceled)

18. A composition comprising a chiral nanostructure prepared by the method of claim 1.

19. The composition of claim 18, wherein n is even and the nanostructure is levorotatory.

20. The composition of claim 18, wherein n is odd and the nanostructure is dextrorotatory.

21. (canceled)

22. The composition of claim 18, wherein the nanostructure comprises CdS, CdSe, CdTe, or any combination thereof.

23. The composition of claim 18, wherein the nanostructure has an absorption maximum at between about 324 nm and about 500 nm.

24. The composition of claim 18, wherein the nanostructure comprises a two-dimensional nanostructure having a thickness of from about 1.22 nm to about 1.83 nm.

25. The composition of claim 18, wherein the nanostructure comprises a helical shape.

26. An article comprising the composition of claim 18.

27. The article of claim 26, wherein the article comprises a solar cell or a light emitting diode.

28. A method for detecting a target analyte in a biological sample, the method comprising contacting the biological sample with the composition of claim 18 and imaging the sample.

29. The method of claim 28, wherein the biological sample comprises cells, tissues, an organ, or a fluid sample collected from a subject.
